(19) United States
(12) Patent Application Publication
(10) Pub. No.: US 2004/0176291 A1
(43) Pub. Date: Sep. 9, 2004

(54) Methods and compositions for soluble CPG15

(76) Inventors: Elly Nedivi, Brookline, MA (US); Ulrich Putz, Cambridge, MA (US)

Correspondence Address: CLARK & ELBING LLP, 101 FEDERAL STREET, BOSTON, MA, 02110, US

(21) Appl. No.: 10/670,911

(22) Filed: Sep. 24, 2003

Related U.S. Application Data
(60) Provisional application No. 60/413,238, filed on Sep. 24, 2002.

(51) Int. Cl. A61K 38/17

(52) U.S. Cl. 514/12

(57) ABSTRACT

Disclosed herein are compositions of soluble CPG15 and methods for treating conditions of excessive cell death, such as neurological conditions, using such compositions. Compounds that inhibit the activity of soluble CPG15 are also disclosed herein for the treatment of conditions of undesirable cell survival, such as cancer.

### CROSS REFERENCES TO RELATED APPLICATIONS

This application claims the benefit of U.S. Provisional Application No. 60/413,238, filed Sep. 24, 2002, herein incorporated by reference.

### STATEMENT AS TO FEDERALLY SPONSORED RESEARCH

This invention was made with government support under Grant Number 5-R29-EY11894-06 awarded by National Institutes of Health. The government has certain rights in the invention.

### BACKGROUND OF THE INVENTION

The invention relates to compositions of soluble CPG15 and methods using soluble CPG15 to treat various conditions, including neurological conditions.

Neurogenesis is an adaptive process whereby a large and excessive population of neurons are initially produced followed by a reduction in the number of neurons as a result of the presence or absence of stimuli from the target organ and the presence or absence of neurotrophic factors in the environment surrounding the neurons. The extensive neuronal remodeling that occurs in response to stimuli both during development and in the adult brain provides the foundation for learning and memory, as well as adaptive reorganization of primary sensory maps. In sum, the proper development of the mature vertebrate nervous systems requires a delicate balance of neuronal cell growth and death.

Many neurological conditions are the result of a shift in the balance towards increased and inappropriate neuronal cell death. For example, the increased death of hippocampal and cortical neurons is responsible for many of the symptoms of Alzheimer's disease (AD); the death of midbrain neurons underlies Parkinson's disease (PD); the death of neurons in the striatum contributes to Huntington's disease (HD); and the death of lower motor neurons results in amyotrohpic lateral sclerosis (ALS). Many other neurological diseases and dysfunctions such as stroke, trauma, spinocerebellar ataxis, and peripheral neuropathies are also characterized by increased cell death.

There are generally two mechanisms by which cell death can occur: apoptosis and necrosis. Necrosis is thought to follow traumatic injury and is characterized by cytoplasmic vacuolization and swelling of the cellular organelles. In necrotic cell death, the plasma membrane lyses, resulting in massive death of groups of cells throughout the affected tissue. Apoptosis, or programmed cell death, is an active process that proceeds via protein synthesis, nuclear fragmentation, chromosome condensation, and activation of proteolytic caspase cascades. Death of a cell by apoptotic pathways does not trigger the death of cells proximal to the apoptotic cell.

The types of cell death involved in neurological conditions varies and, in some cases, has sometimes been hard to classify as necrotic or apoptotic. This is not surprising given that many neurodegenerative diseases are chronic progressive conditions with cell death occurring over a period of five to twenty years or more. In instances of chronic progressive conditions there exists a mixture of necrotic and apoptotic cell death which contributes to the disease progression over time. Even in the case of traumatic injury it is believed that after the initial insult, necrotic cell death occurs, and that this necrosis actually triggers a secondary cascade of apoptotic cell death resulting in a more severe spread of cell damage and death than the damage caused by the initial traumatic injury itself.

The apoptosis pathway is known to play a critical role numerous normal and pathological pathways. For example, apoptosis is directly involved in embryonic development, viral pathogenesis, cancer, autoimmune conditions, and neurodegenerative diseases. There are many features of apoptotic cell death that are shared by a wide variety of cell types.

Emphasis has been placed on understanding the key proteins or factors involved in regulating cell death, particularly apoptosis, in general, and specifically in neuronal cells. Many signals have been identified as initiators of apoptosis in neurons. Extracellular initiation signals include the absence of neurotrophic factors, such as nerve growth factor (NGF) or brain-derived neurotrophic factor (BDNF) in the surrounding environment, activation of a death receptor (e.g., TNF-R/FAS), increased oxidative stress, and the presence of metabolic or environmental toxins. Intracellular initiation signals include disruption of mitochondrial function and the release of mitochondrial factors such as cytochrome c. Following initiation of apoptosis, the activation step takes place. Activation includes proteolytic processing of caspases, an event which allows the caspases to trigger the final step in the process, the effector step. The effector step occurs via the maturation and activation of the effector caspases, again through proteolytic cleavage.

There are many signaling proteins that regulate activation of apoptotic pathways. Examples of these signaling proteins include B-cell lymphoma 2 (Bcl-2) family proteins, caspases (both upstream activator caspases and downstream effector caspases), telomerase, prostate apoptosis response 4 (Par 4), NFκB, inhibitors of apoptosis (IAPs), p53, calcium-binding proteins, to name but a few. Individual proteins either function to promote or inhibit apoptosis, or both. It is the delicate balance between pro-apoptotic and anti-apoptotic factors that results in the dynamic events of neuronal development and remodeling.

A great deal of focus has been placed on identifying genes expressed in neurons that affect this balance between cell growth or survival, and cell death. cpg15 (candidate plasticity gene) was recently identified in a differential screen for genes upregulated by activity in adult hippocampus. CPG15 protein was found to be expressed in differentiated projection neurons within sensory systems throughout the brain, including the auditory system, olfactory system, and visual system. CPG15 is also expressed in the spinal cord and at lower levels outside the central nervous system. In the post-natal and adult brain, peak expression of CPG15 occurs during periods of neuronal dendritic arbor growth and synaptogenesis. In the adult rat, CPG15 is induced in the brain by kainate and in the visual cortex by light. The CPG15 protein has an N-terminal secretion signal characteristic of extracellular proteins, a C-terminal domain comprised of hydrophobic residues indicative of a glycosyl-phosphatidylinositol (GPI) link to the cell surface, and six cysteine residues thought to be critical for correct protein folding (see FIG. 1).

Overexpression studies of full-length CPG15 but not a truncated form lacking the GPI consensus sequence results in an increase in the promotion of dendritic arbor growth and differentiation. Thus, it was concluded that CPG15 is a membrane-bound protein that functions to promote dendritic arbor growth and differentiation.

The role of specific signaling proteins in cell growth and cell death pathways has been studied intensively over the past few years and although several candidate therapeutic targets have been identified, cures for conditions, such as neurological conditions, that are associated with increased cell death, have remained elusive. Given the prevalence of neurological conditions such as AD, PD, HD, and ALS, as well as stroke and trauma, there exists a need for effective therapeutic agents that target the molecules that influence cell death, in particular neuronal cell death. In addition, given the commonalities in apoptotic pathways at the cellular levels, therapeutic agents that are effective for the treatment of neurological conditions are likely to be effective for the treatment of other cell death related conditions.

### SUMMARY OF THE INVENTION

We have discovered a novel soluble, extracellular form of CPG15, hereafter referred to as “s-CPG15”, that we have demonstrated can act as a survival factor by rescuing hippocampal and cortical neurons from multiple types of cell death. We have also discovered that the previously identified biological activity of CPG15, namely the induction of dendritic arbor growth and differentiation, is not a function of the membrane bound form, as was previously believed, but is in fact a function of the novel, soluble form that we have discovered.

Cell death mechanisms in hippocampal and cortical neurons follow classic programmed cell death signaling pathways that are common to additional types of neurons as well as other types of cells. Accordingly, we believe this novel soluble form of CPG15 can be used to promote cell survival in various types of cells where excess apoptosis contributes to disease pathology, including myocytes, liver cells, endothelial cells, hematopoietic cells, bone cells, and immune cells. Conversely, inhibitors of the soluble form of CPG15 can be used to promote cell death in various types of cells where excessive proliferation contributes to disease pathology. Since we show here that s-CPG15 affects classic programmed cell death pathways, the present invention also includes the use of the cpg15 gene and soluble protein as a tool for screening for interacting molecules that induce cell death. Once identified, these molecules can then be used to promote cell death where cell survival and division contribute to the pathologies of diseases, such as cancer, tumor-associated angiogenesis, and conditions resulting from hyperactivity of the immune system.

Accordingly, in a first aspect, the invention features a method of treating or preventing a condition of excessive cell death in a subject. The method includes the steps of administering to a subject a soluble CPG15 compound having s-CPG15 biological activity in an amount and for a time sufficient to prevent, reduce, or eliminate the symptoms of the condition.

In preferred embodiments, the cell death is mediated by apoptosis and can be measured by any standard apoptotic assay such as those described herein. A reduction in cell death typically includes at least a 5% decrease, preferably at least a 10%, 20%, 40%, 50%, 60%, 80%, or 100% decrease in the amount of cell death as compared to a control.

In additional preferred embodiments, the condition is a neurological condition including, but not limited to, any of the following: Alzheimer's Disease, Parkinson's Disease, Huntington's Disease, Amyotrophic Lateral Sclerosis, a condition of the retina and optic nerve, such as retinitis pigmentosa or macular degeneration; traumatic injury to the brain, and stroke. The condition can also be a condition of the bone, skin, muscle, joint, or cartilage; a cardiac condition, such as cardiac ischemia; an autoimmune condition; a liver condition; aging; a condition characterized by ischemia; or an immunodeficiency condition.

In a second aspect, the invention features a method of reducing or preventing cell death, the method comprising administering to a cell s-CPG15 in an amount and for a time sufficient to reduce or prevent said cell death. In preferred embodiments, the cell death is mediated by apoptosis and can be measured by any standard apoptotic assay such as those described herein. A reduction in cell death typically includes at least a 5% decrease, preferably at least a 10%, 20%, 40%, 50%, 60%, 80%, or 100% decrease in the amount of cell death as compared to a control.

In a third aspect, the invention features a method of promoting the survival or differentiation of a cell comprising administering to the cell s-CPG15 for a time and in an amount sufficient to promote the survival or differentiation of the cell. In preferred embodiments the cell is a tissue culture cell and the s-CPG15 is added to the culture media. An increase in the survival or differentiation of the cell is considered at least a 5%, preferably at least 10%, 20%, 40%, 50%, 60%, 80%, or 100% increase in the number of cells surviving or induced to differentiated as compared to a control population as measured using standard assays such as those described herein.

In preferred embodiments of the second and third aspects, the cell is selected from the group consisting of a cell of the nervous system (e.g., a neuron such as a central nervous system neuron, a peripheral nervous system neuron, or a spinal cord neuron), muscle cell, stem cell, immune cell, blood cell, endothelial cell, fibroblast cell, epithelial cell bone cell, skin cell, pancreatic cell, liver cell, cardiomyocyte, oligodendrocyte, and chondrocyte.

In preferred embodiments of any of the methods of the invention, the s-CPG15 lacks either a signal sequence or a GPI linkage sequence or both. In preferred embodiments, the soluble CPG15 lacking a signal sequence and a GPI linkage sequence has the in vitro biological activity of a CPG15 protein wherein (a) the signal sequence and the GPI linkage sequence of the CPG15 protein have been cleaved; (b) the CPG15 protein has been bound to a cell membrane; and (c) the CPG15 protein has been released from the cell. In additional preferred embodiments, the s-CPG15 compound comprises the sequence of SEQ ID NO: 1. The s-CPG15 compound of the invention can also include a post-translational modification. In one example, the post-translational modification includes attachment of any membrane component such as lipids, proteins, phospholipids, or phosphoproteins, or any fragment thereof. Preferred dosages of s-CPG15 include 0.01 μg/kg to about 50 mg/kg per day, preferably 0.01 mg/kg to about 30 mg/kg per day, most preferably 0.1 mg/kg to about 20 mg/kg per day. The s-CPG15 may be given daily (e.g., once, twice, three times, or four times daily) or less frequently (e.g., once every other day, once or twice weekly, or monthly).

In a fourth aspect, the invention features a composition of matter comprising a purified polypeptide having s-CPG15 biological activity.

In preferred embodiments of any of the compositions of the invention, the s-CPG15 lacks either a signal sequence or a GPI linkage sequence or both. In preferred embodiments, the soluble CPG15 lacking a signal sequence and a GPI linkage sequence has the in vitro biological activity of a CPG15 protein wherein (a) the signal sequence and the GPI linkage sequence of the CPG15 protein have been cleaved; (b) the CPG15 protein has been bound to a cell membrane; and (c) the CPG15 protein has been released from the cell. In additional preferred embodiments, the s-CPG15 compound comprises the sequence of SEQ ID NO: 1. The s-CPG15 compound of the invention can also include a post-translational modification. In one example, the post-translational modification includes attachment of any membrane component such as lipids, proteins, phospholipids, or phosphoproteins, or any fragment thereof.

In a fifth aspect, the invention features a method of treating or preventing a condition of undesirable cell survival in a subject comprising administering to the subject a purified antibody or antigen-binding fragment that specifically binds a polypeptide having s-CPG15 biological activity for an amount and for a time sufficient to prevent, reduce, or eliminate the symptoms of the condition. In preferred embodiments, the condition is cancer, tumor-associated angiogenesis, or an immune system condition.

In a sixth aspect, the invention features a method of treating or preventing a condition of undesirable cell survival in a subject, comprising administering to the subject an antisense nucleobase oligomers complementary to a nucleic acid sequence encoding a protein having s-CPG15 biological activity, in an amount and for a time sufficient to reduce or inhibit the expression of s-CPG15. In preferred embodiments, the condition is cancer, tumor-associated angiogenesis, or an immune system condition. Desirable, the antisense nucleobase oligomers is 8 to 30 nucleotides in length.

In a seventh aspect, the invention features a method of treating or preventing a condition of undesirable cell survival in a subject, comprising administering to the subject a double stranded RNA that is complementary to an mRNA sequence encoding a protein having s-CPG15 biological activity, in an amount and for a time sufficient to reduce or inhibit the expression of s-CPG15. In preferred embodiments, the condition is cancer, tumor-associated angiogenesis, or an immune system condition. Desirably, the ds RNA is processed into small interfering RNAs 19 to 25 nucleotides in length or is a short hairpin RNA. Preferred short hairpin RNAs include the sequence of SEQ ID NO: 2 or SEQ ID NO: 3.

In an eighth aspect, the invention features a method of treating or preventing a condition of undesirable cell survival in a subject, comprising administering to the subject a nucleic acid encoding a truncated form of s-CPG15 in an amount and for a time sufficient to reduce or inhibit the biological activity of s-CPG15. In preferred embodiments, the condition is cancer, tumor-associated angiogenesis, or an immune system condition. In a related aspect, the invention features a method of treating or preventing a condition of undesirable cell survival in a subject, comprising administering to the subject a compound comprising a truncated form of s-CPG15 in an amount and for a time sufficient to reduce or inhibit the biological activity of s-CPG15.

In preferred embodiments of the above aspects, a reduction in undesirable cell survival includes at least a 5% decrease, preferably at least a 10%, 20%, 40%, 50%, 60%, 80%, or 100% decrease in the amount of cells as compared to a control when measured by standard art-known cell survival assays such as those described herein.

In additional aspects, the invention features a method of enhancing cell death comprising administering to a cell an antibody or antigen-binding fragment that specifically binds s-CPG15; an antisense nucleobase oligomers complementary to a nucleic acid sequence encoding a protein having s-CPG15 biological activity; a double stranded RNA that is complementary to an mRNA sequence encoding a protein having s-CPG15 biological activity; or a truncated form of CPG15. Any or all of the above are administered in an amount sufficient to reduce or inhibit the biological activity of s-CPG15 or to enhance cell death. The above aspects can be used to treat any condition characterized by undesirable cell survival (e.g., cancer, tumor-associated angiogenesis, or an immune system condition). In any of the above aspects, cell death is measured by standard apoptotic assays such as those described herein and a increase in cell death is at least a 5% increase, preferably at least a 10%, 20%, 40%, 50%, 60%, 80%, or 100% increase in the number of cells undergoing apoptosis.

The invention also features a composition of matter comprising a purified antibody or antigen-binding fragment thereof that specifically binds s-CPG15. The antibody or antigen-binding fragment thereof can be a monoclonal antibody or a polyclonal antibody. Monoclonal and polyclonal antibodies may be produced by methods known in the art. These methods include the immunological method described by Kohler and Milstein (Nature, 256: 495-497, 1975) and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science, 246, 1275-1281, 1989).

Monoclonal antibodies may be prepared from supernatants of cultured hybridoma cells or from ascites induced by intra-peritoneal inoculation of hybridoma cells into mice. The hybridoma technique described originally by Kohler and Milstein (Eur. J. Immunol, 6, 511-519, 1976) has been widely applied to produce hybrid cell lines that secrete high levels of monoclonal antibodies against many specific antigens.

The invention also features a composition of matter comprising an siRNA that is complementary to an mRNA sequence encoding s-CPG15. Preferred siRNAs include SEQ ID NO: 2 and SEQ ID NO: 3.

Any of the compositions of the invention are preferably formulated with a pharmaceutically acceptable excipient.

The invention also features a method of manufacturing s-CPG15 comprising expressing the s-CPG15 protein in a population of cells and isolating the s-CPG15 from the supernatant of the cell population. In preferred embodiments, the s-CPG15 is at least 85% pure and the cells are neuronal cells or hippocampal cells; COS cells; CV-1 cells; L cells; C127 cells; 3T3 cells; CHO cells; HeLa cells; 293 cells; 293T cells; and BHK cells. In preferred embodiments, the s-CPG15 lacks either a signal sequence or a GPI linkage sequence or both. In preferred embodiments, the soluble CPG15 lacking a signal sequence and a GPI linkage sequence has the in vitro biological activity of a CPG15 protein wherein (a) the signal sequence and the GPI linkage sequence of the CPG15 protein have been cleaved; (b) the CPG15 protein has been bound to a cell membrane; and (c) the CPG15 protein has been released from the cell. In additional preferred embodiments, the s-CPG15 compound comprises the sequence of SEQ ID NO: 1. The s-CPG15 compound of the invention can also include a post-translational modification. In one example, the post-translational modification includes attachment of any membrane component such as lipids, proteins, phospholipids, or phosphoproteins, or any fragment thereof.

In addition, the invention features a method of identifying candidate compounds that regulate cell death, cell survival, or cellular differentiation pathways. The method includes the steps of (a) mixing s-CPG15 with a test mixture and (b) identifying a candidate compound in the test mixture that interacts with the s-CPG15. In preferred embodiments, the method also includes the step after step (a) of incubating the s-CPG15/test mixture with an insoluble affinity support reagent that specifically binds s-CPG15. In additional preferred embodiments the method also includes the step of recovering the affinity support reagent bound to s-CPG15. The test mixture can be a cell lysate or a lysate from a tissue. The modulation of cell death can be an increase or a decrease as measured by standard apoptosis, cell survival, or cell differentiation assays such as those described herein. Preferably the increase or decrease will result in a change of at least 5%, more preferably at least 10%, 20%, 40%, 50%, 60%, 80%, or 100%.

In preferred aspects of any of the methods of the invention, a subject includes humans and other animals, preferably warm-blooded mammals including mice, rats, guinea pigs, hamsters, rabbits, cats, dogs, goats, sheep, cows, or monkeys.

By “candidate plasticity gene 15 (cpg15)” is meant a gene encoding a protein that has an N-terminal secretion signal sequence, a C-terminal domain comprised of hydrophobic residues diagnostic of a gycosyl-phosphatidylinositol (GPI) link to the cell surface, and six cysteine residues postulated to be critical for correct protein folding. cpg15 encodes both membrane bound CPG15 and s-CPG15. CPG15 is also sometimes referred to as neuritin. As used herein, “CPG15” is used to refer to the complete, unprocessed form of the protein including the GPI consensus sequence and the secretion signal sequence. CPG15 can also refer to the membrane bound form of the protein which is a naturally secreted, processed form of the protein that lacks the signal sequence and that has a cleaved GPI sequence but is anchored to the plasma membrane via a lipid moiety.

“cpg15 ” includes any nucleic acid sequence that encodes a protein that is substantially identical to any of the following: rat CPG15/neuritin (Nedivi et al., Proc. Natl. Acad. Sci. USA, 93:2048-2053, 1996; Hevroni et al., J. Mol. Neurosci. 10:75-98, 1998; GenBank accession number U88958), mouse CPG15 (GenBank accession number BC03553 1); human CPG15/neuritin (Naeve et al., Proc. Natl. Acad. Sci., USA, 94:2648-2653, 1997; GenBank accession number NM016588 and AF136631), Xenopus CPG15 (J. Comp. Neurol., 435:464-473, 2001; GenBank accession number AF378092), and cat CPG15 (Corriveau et al., J. Neurosci., 19:7999-8008, 1999). By “substantially identical” is meant a protein or nucleic acid exhibiting at least 50%, preferably 85%, more preferably 90%, and most preferably 95% identity to a reference protein or nucleic acid sequence. For proteins, the length of comparison sequences will generally be at least 16 amino acids, preferably 20 amino acids, more preferably at least 25 amino acids and most preferably at least 35 amino acids. For nucleic acids the length of comparison sequences will generally be at least 50 nucleotides, preferably at least 60 nucleotides, more preferably at least 75 nucleotides and most preferably 110 nucleotides or more.

Methods to determine identity are available in publicly available computer programs. Computer program methods to determine identity between two sequences include, but are not limited to, the GCG program package (Devereux et al., Nucleic Acids Research 12: 387, 1984), BLASTP, BLASTN, and FASTA (Altschul et al., J. Mol. Biol. 215:403 (1990). The well-known Smith Waterman algorithm may also be used to determine identity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). These software programs match similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “soluble cpg15 (s-CPG15)” is meant a form of CPG15 that is naturally secreted from the cell, is soluble, and has the ability to promote cell survival or protect a cell from cell death. s-CPG15 generally refers to the core domain of the protein after cleavage of the GPI linkage sequences or the secretion signal sequence, or both. These sequences are typically cleaved off after translation and processing of the protein. The resulting core domain s-CPG15 sequence includes the following sequence: AGKCDAVFKGFSDCLLKLGDSMANYPQGLDDKTNIKTVCTYWEDFHSCTVTAL TDCQEGAKDMWDKLRKESKNLNIQGSLFELCGSG (SEQ ID NO: 1). An asparagine residue (N) can also be present at the carboxy terminus of the protein.

In general, the soluble form of CPG15 is produced via cleavage of the membrane bound form of CPG15 causing release of the protein from the membrane into the extracellular space. s-CPG15 is typically purified from the supernatant of growing cells.

In preferred embodiments, s-CPG15 includes modifications to the protein (e.g., the carboxy-terminus of the protein) that occur after localization to the plasma membrane. Such modifications can include, for example, post-translational modifications to the protein including but not limited to phosphorylation, hydroxylation, sulfation, acetylation, glycosylation, subunit dimerization or multimerization, and cross-linking or attachment to any other proteins, or fragments thereof, or membrane components, or fragments thereof (e.g., cleavage of the protein from the membrane with a lipid component attached).

By “s-CPG15 biological activity” is meant the ability to promote cell survival or to prevent or reduce cell death. The biological activity of s-CPG15 can be assayed using standard apoptotic assays such as the cell starvation assays as described herein. In addition, s-CPG15 biological activity includes the ability to promote growth and differentiation. These functions can also be measured using standard art known assays such as those described herein. One potential assay to measure neuronal cell growth and differentiation is using the in vitro explant assay for process outgrowth as described in Placzek et al., (Science 250:985-988, 1990); Ringstedt et al., (J. Neurosci. 20:4983-4991, 2000); Charron et al., (Cell 113:11-23, 2003); and Wang et al., (Nature 401:765-769, 1999). Tissue sources used in these assays can also be obtained using the methods described in Baranes et al., (Proc. Natl. Acad. Sci. 93:4706-4711, 1996). “In vitro biological activity” can also be measured using the cell starvation assays described herein or the in vitro explant assay for process outgrowth. Soluble CPG15 will preferably rescue at least 10% of the cells from starvation-induced apoptosis; more preferably at least 20%, 30%, 50%, 75% or more. Preferably, a soluble CPG15 sample, will contain less than 20%, more preferably less than 10%, 5% or 1% CPG15.

By “truncated CPG15 (t-CPG15)” is meant a non-natural form of CPG15 that lacks the amino acids encoding the GPI linkage sequence (i.e., t-CPG15 includes only amino acids 1 -114 or 1 -115 of the human CPG15 protein (GenBank Accession Number NM_{—}016588)). In general, t-CPG15 is expressed from an engineered construct containing the nucleic acid sequence encoding CPG15 but lacking the nucleotides that encode the GPI linkage sequence. This truncated form of CPG15 does not follow the GPI linkage pathway but is instead secreted directly out of the cell without membrane attachment or modifications associated with membrane attachment. t-CPG15 can function as a dominant negative to inhibit s-CPG15 biological activity.

By “membrane component” is meant any lipid, protein, phospholipid, or phosphoprotein or any fraction thereof that is found in a cell membrane.

By “antisense nucleobase oligomer” is meant a nucleobase oligomer, regardless of length, that is complementary to the coding strand or MRNA of a gene that encodes a protein having s-CPG15 biological activity. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified (e.g., phosphorothiates, phosphorodithiates, and phosphotriesters) and unmodified, oligonucleotides with modified (e.g., morpholino linkages and heteroatom backbones) or unmodified backbones, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. patent application Ser. Nos. 20030114412 and 20030114407, incorporated herein by reference. The nucleobase oligomer can also be targeted to the translational start and stop sites. Preferably the antisense nucleobase oligomer comprises from about 8 to 30 nucleotides. The antisense nucleobase oligomers can also contain at least 40, 60, 85, 120, or more consecutive nucleotides that are complementary to the mRNA or DNA that encodes a protein having s-CPG15 biological activity, and may be as long as the full-length mRNA or gene.

By “small interfering RNAs (siRNAs)” is meant an isolated dsRNA molecule, preferably greater than 10 nucleotides in length, more preferably greater than 15 nucleotides in length, and most preferably greater than 19 nucleotides in length, that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nucleotide RNA or internally (at one or more nucleotides of the RNA). In a preferred embodiment, the RNA molecules contain a 3'hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNAi. As used herein “mediate RNAi” refers to the ability to distinguish or identify which RNAs are to be degraded.

Desirably, the antisense nucleobase oligomers or siRNA used for RNA interference will cause an overall decrease preferably of 20% or greater, more preferably of 50% or greater, and most preferably of 75% or greater, in the level of protein or nucleic acid, detected by standard art known assays, as compared to samples not treated with antisense nucleobase oligomers or dsRNA used for RNA interference. Examples of assays for protein expression include western blotting, examples of assays for RNA expression include northern blotting, PCR, and RNase protection assays, and examples of assays for DNA expression include Southern blotting and PCR.

By “cell death” is meant the process or series of events, which ultimately lead to a non-functioning, non-living cell. Cell death as used herein typically refers to apoptosis (programmed cell death) or necrosis. By “preventing or reducing” cell death is meant any treatment or therapy that causes an overall decrease in the number of cells undergoing cell death relative to a control. Preferably, the decrease will be at least 15%, more preferably at least 25%, and most preferably at least 50%. By “excessive cell death” is meant an increase in the number of cells undergoing cell death as compared to a control population of cells. Preferably, excessive cell death includes an increase of 10% or more in the total number of cells undergoing cell death. More preferably the increase is 15%, 20%, 25% or more, and most preferably an increase of 40% or more in the total number of cells undergoing cell death as compared to a control population of cells.

By “apoptosis” or “apoptotic cell death” is meant the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of neurodegenerative diseases or neurogenesis), cardiomyocytes (e.g., after myocardial infarction or over the course of congestive heart failure), immune cells (e.g., after HIV infection), and cancer cells (e.g., after exposure to radiation or chemotherapeutic agents).

By “necrosis” or “necrotic cell death” is meant cell death associated with a passive process involving loss of integrity of the plasma membrane and subsequent swelling, followed by lysis of the cell.

By “cell survival” is meant the reversal or prevention of cell death signaling pathways or the promotion of pathways that antagonize cell death, thereby increasing the life span of a cell or the number of cells that survive in a given situation, relative to a control. By “promoting” cell survival is meant any treatment or therapy that causes an overall increase in the number of cells. Preferably, the increase will be at least 15%, more preferably at least 25%, and most preferably at least 50%. “Undesirable cell survival” is characterized by a increase in cell proliferation or a decrease in cell death such that the total number of growing cells exceeds that of a normal control population. Preferably, “undesirable cell survival” refers to an increase of 15% or more in the total number of growing cells. More preferably the increase is 25% or more and most preferably the total number of growing cells will be 50% or more than the number of growing cells in a control population. Preferably, changes in cell survival and cell death are measured using a standard serum starvation assay such as the one described herein below.

By “differentiation” is meant the process during which young, immature (unspecialized) cells take on individual characteristics and reach their mature (specialized) form and function. By “promoting” cell differentiation is meant any treatment or therapy that causes an overall increase in the number of differentiated cells as measured by assays which quantitate the presence or absence of a defining characteristic of a differentiated cell. Preferably, the increase in differentiation of a cell population will be at least 15%, more preferably at least 25%, and most preferably at least 50%. In one example, stem cell conversion to neurons can be measured by expression of neuronal markers such as neurofilament-M, Map2, and neuron specific enolase. In another example, the clonogenic Colony Assay offered by Cambrex Corporation, can be used to determine differentiation of hematopoietic progenitor cells into myeloid (CFU-GM), erythroid (CFU-E, BFU-E), megakaryocyte (CFU-Meg), and mixed (myeloid and erythroid) colonies.

By “specifically binds” is meant an antibody or antigen binding fragment thereof that recognizes and binds an antigen but that does not substantially recognize or bind to other molecules in a sample, e.g., a biological sample, that naturally includes protein. Specific recognition of an antigen by an antibody can be assayed using standard art known techniques such as immunoprecipitation, western blotting, and ELISA. An antibody or antigen-binding fragment thereof that specifically binds s-CPG15 does not recognize membrane bound CPG15.

By “neurological condition” is meant any condition of the central or peripheral nervous system that is associated with neuron degeneration or damage. Specific examples of neurological conditions include, but are not limited to, Alzheimer's disease, Parkinson's disease, Huntington's disease, ALS, peripheral neuropathies, stroke, trauma, and other conditions characterized by neuronal death or loss of neurons, whether central peripheral, or motor neurons. Neuronal conditions also include conditions of the retina and optic nerve such as macular degeneration, retinal degeneration, retinitis pigmentosa, and general macular dystrophies

By “treating” is meant administering a compound or a pharmaceutical composition for prophylactic and/or therapeutic purposes. To “treat disease” or use for “therapeutic treatment” refers to administering treatment to a subject already suffering from a condition to improve the subject's condition. Preferably, the subject is diagnosed as suffering from a condition based on identification of any of the characteristic symptoms known for that condition. To “prevent disease” refers to prophylactic treatment of a subject who is not yet ill, but who is susceptible to, or otherwise at risk of, developing a particular condition. Thus, in the claims and embodiments, treating is the administration to a subject either for therapeutic or prophylactic purposes.

By “operably linked” is meant that a gene and a regulatory sequence(s) are connected in such a way as to permit gene expression when the appropriate molecules (e.g., transcriptional activator proteins) are bound to the regulatory sequence(s).

Other features and advantages of the invention will be apparent from the following description of the preferred embodiments thereof, and from the claims.

### BRIEF DESCRIPTION OF THE DRAWINGS

The application file contains drawings executed in color (FIGS. 3A-3H, 4A-4D, 7, 9A, 1OA-1OK, 11A-D, and 11L-11O). Copies of this patent or patent application with color drawings will be provided by the Office upon request and payment of the necessary fee.

FIG. 1 shows a schematic of the full-length CPG15 protein with the secretion signal (SS), the CPG15 core domain and the consensus sequence for a GPI-linkage (GPI). The SS and GPI sequences are cleaved off during processing such that the CPG15 core domain becomes attached to the outer cell membrane through a GPI-anchor.

FIG. 2 shows the tissue specific expression of cpg15 by reverse transcriptase-polymerase chain reaction (RT-PCR).

FIGS. 3A-3H show cpg15 in situ hybridizations on coronal sections through the telencephalon during prenatal development of rat brains. FIGS. 3I-3J show in situ hybridizations on saggital sections through postnatal rat brains. Ages of rats are designated on the left. Dark field photomicrographs of embryonic times are shown in the left column, paired on the right with bright-field pictures of toluidine blue stained sections overlayed with their dark-field views. Abbreviations: nascent cortical plate (CP), retina (RET), subventricular zone (SVZ), dorsal thalamus (DT), hippocampus (HI), proliferative external granular layer (EGL), third ventricle (3V), lateral ventricle (LV), olfactory epithelium (OE), ganglionic eminence (GE), trigeminal ganglion (TG), neocortex (CTX), sensory thalamus (sTH), inferior colliculus (IC), and superior colliculus (SC). Scale bars for FIGS. 3A-3H are 0.5 μm and for FIGS. 3I-3J are 1 mm.

FIGS. 4A-4D show the transfer of s-CPG15 from transfected cells to non-transfected cells. FIG. 4A shows a schematic of the CPG15-FLAG IRES EGFP construct; IRES, internal ribosome entry site, EGFP, enhanced green fluorescent protein. FIG. 4B shows 293T cells transfected with CPG15-Flag IRES EGFP. Transfected cells show a green EGFP signal (left panel), CPG15-FLAG is stained with Rhodamine (red, center panel), and a merged view is shown in the right panel. Arrowheads indicate a transfected cell while arrows indicate a non-transfected cell. FIG. 4C shows 293T cells transfected separately with CPG15-FLAG IRES EGFP or a plasmid expressing red fluorescent protein (RFP). Cells were mixed and stained for CPG15-FLAG (Cy5, blue). The arrow indicates a non-transfected cell staining positively for CPG15-FLAG. FIG. 4D shows 293T cells transfected with CPG15-FLAG IRES EGFP or a plasmid expressing RFP. Cells were co-cultured on the same coverslip without contact and stained for CPG15-FLAG (Cy5, blue).

FIG. 5 shows the two different forms of CPG15. 293T cells were transfected with CPG15-FLAG IRES EGFP and treated with Phospholipase C (PLC; “+PLC”) or left untreated (“-PLC”). Cell extracts and supernatants were collected, immunoprecipitated with an anti-FLAG antibody, and western-blotted for CPG15-FLAG.

FIG. 6 shows the presence of CPG15 in the soluble fraction taken from E14 and E18 brains, and adult cortices probed with an antibody against CPG15. Staining for the transferrin (Tf) receptor and the enzyme Akt served as membrane- and soluble-fraction controls, respectively.

FIG. 7 shows a schematic depicting the differences between a healthy cell undergoing apoptosis (steps 1-5) and necrosis (steps 1′ and 2′). The blue insets show chromatin staining of nuclei from a healthy, an apoptotic, and a necrotic neuron (stained with Hoechst 33342).

FIG. 8 shows a schematic of the starvation assay using primary hippocampal or cortical neurons.

FIGS. 9A-9B shows the prevention of starvation-induced apoptosis by supernatant containing s-CPG15. Primary hippocampal neurons were cultured for six days and then starved in the absence or presence of supernatant from 293T cells overexpressing CPG15-FLAG or from 293T cells transfected with vector alone. After 12 hours, medium was changed back to normal feeding media for an additional 12 hours. Neurons were then stained with Hoechst 33342 (FIG. 9A) and the results were quantitated (FIG. 9B).

FIGS. 10A-10K shows the prevention of starvation-induced apoptosis by purified s-CPG15. FIGS. 10A and 10B show the increase in the number of fragmented nuclei seen with Hoechst staining (marked by arrows) after growth factor deprivation (starvation). FIG. 10C shows the prevention of starvation-induced increase in fragmented nuclei by the addition of s-CPG15. FIG. 10D shows the quantification of FIGS. 10A-10C. Starvation significantly increased the percent of apoptotic neurons (* p<0.002), and s-CPG15 application prevented this increase (p<0.003). FIGS. 10E and 10F show the increased numbers of neurons immunostained with an antibody against cleaved caspase 3 indicative of starvation induced apoptosis. FIG. 10G shows the prevention of the increase in cleaved caspase 3 immunopositive cells by the addition of purified CPG15 to starved neurons. FIG. 10H shows the quantification of FIG. 10E-10G. Starvation increased the percent of cleaved caspase 3 immunopositive neurons (p<0.001) and s-CPG15 application prevented this increase (p<0.002). FIGS. 10I-10K show an overlay of FIGS. 10A and 10E, FIGS. 10B and 10F, and FIGS. 10C and 10G indicating that cells with fragmented nuclei scored by Hoechst staining contain activated caspase 3. Scale bar 10 μm.

FIGS. 11A-11Q show the depletion of endogenous CPG15 using lentivirus delivered cpg15-shRNA increases apoptosis of cortical neurons in vitro and in vivo. FIG. 11A-11D show the immunohistochemistry staining of cultured cortical neurons infected with CPG15-FLAG lentivirus also expressing EGFP, alone (FIGS. 11A and 11B) or together with a cpg15-shRNA-lentivirus (FIGS. 11C and 11D). CPG15 immunocytochemistry is shown in red, EGFP (green) marks infected cells. FIG. 11D shows that infection with the cpg15-shRNA-lentivirus reduces expression of CPG15FLAG, but not EGFP. FIG. 11E shows western blots of cell extracts (cells) and supernatants (sup.) from cultured cortical neurons infected as indicated, harvested four days later, and immunoprecipitated with an anti-FLAG antibody. FIGS. 11F-11Q show intraventricular injection of cpg15-shRNA-lentivirus increases apoptosis of embryonic cortical neurons in vivo. FIGS. 11F and 11G show Nissl stained coronal hemi-sections from brains infected with GFP-lentivirus. FIGS. 11H-11J show Nissl stained coronal hemi-sections from brains infected with cpg15-shRNA-lentivirus. Brains are shown so that low to high infection levels are from left to right. FIG. 11K shows a scatter plot summarizing the ventricular size for EGFP- or cpg15-shRNA-lentivirus infected brains. Each circle represents one hemisphere. The boxes in FIGS. 11G and 11H are shown at higher magnification than in FIGS. 11L and 11M, and FIGS. 11N and 11O, respectively. FIGS. 11L and 11N show TUNEL staining. FIGS. 11M and 11O show overlay of TUNEL staining and EGFP staining. FIGS. 11P and 11Q show quantification of TUNEL positive cells in the neocortex (FIG. 11P) or diencephalon (FIG. 11Q) of uninfected brains and brains infected with EGFP-lentivirus or cpg15shRNA-lentivirus (* p<0.003). Scale bars: (FIG. 11D) 25 μm, (FIG. 11J) 1 mm, (FIG. 11O) 0.1 mm.

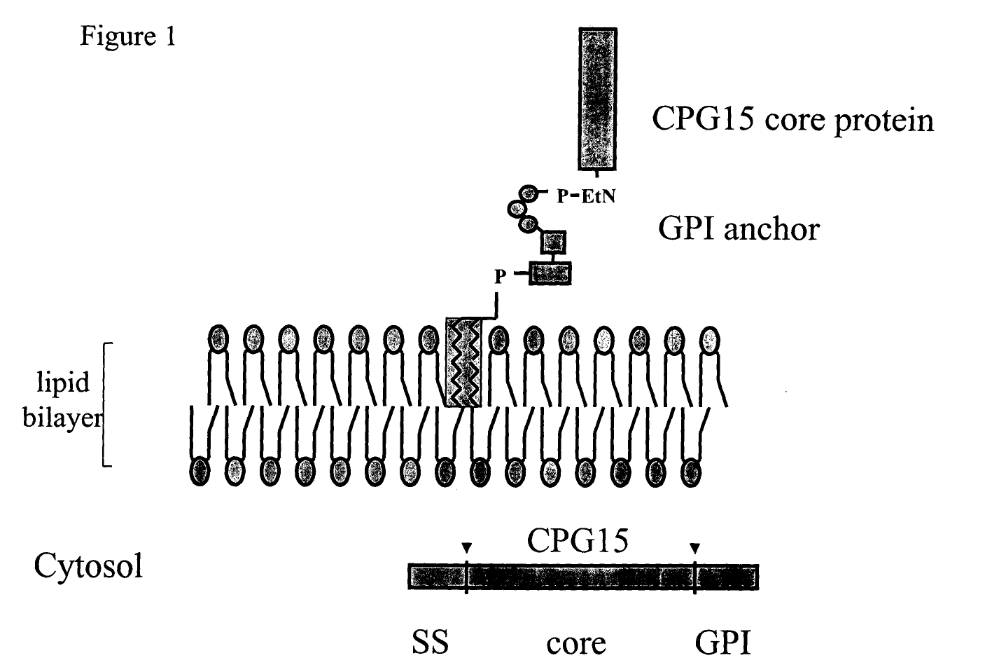

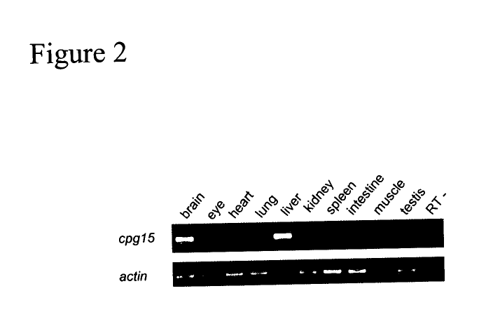

### DETAILED DESCRIPTION

Many diseases result from increased cell death, including neurodegenerative conditions, cardiac conditions, muscle conditions, liver conditions, bone conditions, skin conditions, and autoimmune diseases. There is a need for effective compounds that can prevent or reduce cell death and can therefore be used as therapies for the treatment of such diseases. We have discovered s-CPG15, a soluble form of CPG15, which can promote cell survival in hippocampal and cortical neurons. Cell death pathways are conserved among various types of cells; therefore s-CPG15 can be used as a compound to promote cell survival in neurons in general, as well as additional cell types. We have also discovered that s-CPG15 can promote cell growth and differentiation and can therefore be used in applications such as promoting the differentiation of stem cells. In addition, inhibitors of s-CPG15 can be used to treat conditions which result from undesirable cell survival. Diseases that result from undesirable cell survival include any form of cancer, tumor-associated angiogenesis, and conditions resulting from hyperactivity of the immune system. By preventing or reducing the biological activity of s-CPG15, inhibitors of s-CPG15 can be used to promote apoptosis in cells.

Preparation of Purified s-CPG15

We have discovered soluble CPG15 which is a secreted product that is no longer membrane bound. We have discovered that soluble CPG15 acts as a survival factor by preventing cell death. In addition to this newly described activity of promoting survival, we have also discovered that it is the soluble form of CPG15 that possesses the process outgrowth and differentiation promoting functions previously attributed to the membrane bound form of CPG15.

s-CPG15 includes any amino acid sequence that is substantially identical to the full-length CPG15 sequence after processing (GenBank accession numbers provided above), and results in a soluble form of the protein that is secreted outside the cell and is capable of promoting survival by providing protection from cell death. s-CPG15 is also capable of promoting neuronal growth and differentiation. Soluble CPG15 does not refer to the previously reported membrane bound CPG15, the full-length, unprocessed CPG15, or the truncated form of CPG15. Analogs or homologs ofs-CPG15, which retain the biological activity of s-CPG15, are also included and can be constructed, for example, by making various substitutions of residues or sequences, deleting terminal or internal residues or sequences not needed for biological activity, or adding terminal or internal residues which may enhance biological activity. Amino acid substitutions, deletions, additions, or mutations can be made to improve expression, stability, or solubility of the protein in the various expression systems. Generally, substitutions are made conservatively and take into consideration the effect on biological activity. Mutations, deletions, or additions in nucleotide sequences constructed for expression of analog proteins or fragments thereof must, of course, preserve the reading frame of the coding sequences and preferably will not create complementary regions that could hybridize to produce secondary mRNA structures such as loops or hairpins which would adversely affect translation of the mRNA.

s-CPG15 analogs can also include any post-translationally modified forms. Examples of post-translational modifications include but are not limited to phosphorylation, glycosylation, hydroxylation, sulfation, acetylation, isoprenylation, proline isomerization, subunit dimerization or multimerization, and cross-linking or attachment to any other proteins, or fragments thereof, or membrane components, or fragments thereof (e.g., cleavage of the protein from the membrane with a membrane lipid component attached).

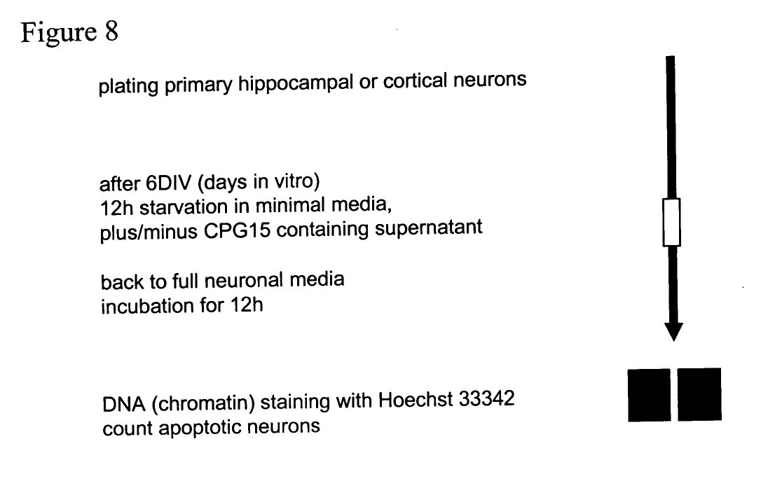

The biological activity of s-CPG15 or any homologs, analogs or mutants thereof can be determined, for example, by cell growth or cell death assays such as the serum starvation assays depicted in FIG. 8. In this assays, cells, preferably hippocampal cells, are grown in the absence of B27 (serum substitute; GIBCO-BRL) for a period of time long enough to initiate cell death. s-CPG15, or inhibitors or enhancers of s-CPG15 biological activity, are then added to the cells and the promotion of cell survival is measured by a reduction in the number of cells undergoing cell death, as measured by any of the assays described below. The biological activity of s-CPG15 can also be measured, for example, by in vitro explant assays for process outgrowth such as those described by Placzek et al., supra, Ringstedt et al., supra, Charron et al., supra, Wang et al., supra.

The s-CPG15 is preferably produced by recombinant DNA methods by inserting a DNA sequence encoding cpg15, or fragments thereof, or analogs thereof into a recombinant expression vector and expressing the DNA sequence under conditions promoting expression. In one example, a cDNA encoding full length cpg15 is used to produce s-CPG15. General techniques for nucleic acid manipulation are described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience:New York, 1987) and periodic updates. The DNA encoding cpg15 is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

Recombinant proteins can also be produced using methodologies for activating endogenous genes by positioning an exogenous regulatory sequence at various positions ranging from immediately adjacent to the gene of interest to 30 kilobases or further upstream of the transcribed region of an endogenous gene. Such methods are described, for example, in U.S. Pat. No. 5,641,670, 5,733,761, and 5,272071; WO 91/06666; WO 91/06667; and WO 90/11354, all of which are incorporated herein by reference.

The recombinant DNA can also include any type of protein tag sequence which may be useful for identifying the protein. Examples of protein tags include but are not limited to a histidine tag, a FLAG tag, a myc tag, or a GST tag. Appropriate cloning and expression vectors for use with bacterial, fungal, yeast, and mammalian cellular hosts can be found in Cloning Vectors: A Laboratory Manual, (Elsevier, N.Y., 1985), the relevant disclosure of which is hereby incorporated by reference.

The expression construct is introduced into the host cell using a method appropriate to the host cell, as will be apparent to one of skill in the art. The expression construct can be introduced for transient expression of the protein or stable expression by selecting cells using a selectable marker in order to generate a stable cell line that expresses the protein continuously. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells for expression of s-CPG15 from recombinant vectors include prokaryotes, yeast, mammalian cells, or amphibian cultured neurons under the control of appropriate promoters. Cell-free translation systems can also be employed to produce proteins using RNAs derived from the DNA constructs disclosed herein. Prokaryotes include gram negative or gram positive organisms, for example, E. coli or Bacillus spp. Prokaryotic expression hosts may be used for expression of s-CPG15 or analogs thereof that do not require extensive proteolytic and disulfide processing. s-CPG15 may also be expressed in yeast, preferably from the Saccharomyces species, such as S. cerevisiae.

Various mammalian or insect cell culture systems can also be employed to express recombinant protein. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines.

Purified s-CPG15 or analogs thereof are prepared by culturing suitable host/vector systems to express the recombinant proteins. In a preferred embodiment, the full-length cpg15 is expressed, targeted to the membrane, via the secretion signal, where it is anchored, via the GPI anchor, and then cleaved off of the membrane to release the soluble form. The protein is likely to be released from the membrane with an additional membrane or protein component such as a membrane lipid. The protein is then purified from culture media or cell extracts.

In one example, supernatants from systems which secrete recombinant protein into culture media can be first concentrated using a commercially available protein concentration filter, for example, an Amicon or Millipore Pellicon ultrafiltration unit.

Following the concentration step, the concentrate can be applied to a suitable purification matrix. For example, a suitable purification matrix can comprise a counter structure protein, lectin, or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose, or other types of matrices commonly employed in protein purification. Alternatively, a cation exchange step can be employed. Suitable cation exchangers include various insoluble matrices comprising sulfopropyl or carboxymethyl groups. Gel filtration chromatography also provides a means of purifying the s-CPG15.

Affinity chromatography is a particularly preferred method of purifying s-CPG15 and analogs thereof. For example, s-CPG15 expressed as a fusion protein comprising an immunoglobulin Fc region can be purified using Protein A or Protein G affinity chromatography. Monoclonal antibodies against the s-CPG15 protein may also be useful in affinity chromatography purification, by utilizing methods that are well-known in the art. A tagged version of the protein, such as a FLAG tagged version, can also be expressed and purified using antibodies directed to the tag sequence. In general, affinity chromatography will be performed using the soluble cellular fraction, or, in the case of tissue culture cells, the supernatant.

Finally, one or more reversed-phase high performance liquid chromatography (RP-HPLC) steps employing hydrophobic RP-HPLC media, e.g., silica gel having pendant methyl or other aliphatic groups, can be employed to further purify a s-CPG15 composition. Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. High performance liquid chromatography (HPLC) can be employed for final purification steps. Microbial cells employed in expression of recombinant protein can be disrupted by any convenient method, including freeze-thaw cycling, sonication, mechanical disruption, or use of cell lysing agents.

Fermentation of yeast which express s-CPG15 greatly simplifies purification. Secreted recombinant protein resulting from a large-scale fermentation can be purified by methods analogous to those disclosed by Urdal et al. (J. Chromatog. 296:171, 1984). This reference describes two sequential, reversed-phase HPLC steps for purification of recombinant human GM-CSF on a preparative HPLC column.

Protein synthesized in recombinant culture is characterized by the presence of cell components, including proteins, in amounts and of a purity which depend upon the purification steps taken to recover the inventive protein from the culture. These components ordinarily will be of yeast, prokaryotic or non-human higher eukaryotic origin and preferably are present in innocuous contaminant quantities, on the order of less than about 1 percent by weight.

In addition to the methods employing recombinant DNA, s-CPG15 can be purified from sources that naturally produce the soluble form of the protein. Examples of these sources include neuronal cells and brain lysates isolated from mouse or rat brain after seizures. In particular, it is preferred that the brain lysate predominantly includes the hippocampus and the cerebral cortex. The s-CPG15 from these sources can be purified and concentrated using any of the methods described above.

After purification, s-CPG15 may be exchanged into different buffers and/or concentrated by any of a variety of methods known to the art, including, but not limited to, filtration and dialysis. The purified s-CPG15 is preferably at least 85% pure, more preferably at least 95% pure, and most preferably at least 98% pure. Regardless of the exact numerical value of the purity, the s-CPG15 is sufficiently pure for use as a pharmaceutical product.

S-CPG15 proteins, particularly short fragments of the protein which retain s-CPG15 biological activity can also be produced by chemical synthesis (e.g., by the methods described in Solid Phase Peptide Synthesis, 2^{nd} ed., 1984, The Pierce Chemical Co., Rockford, Ill.). Modifications to the protein can also be produced by chemical synthesis.

Treatment of Conditions Involving Inappropriate Cell Death

The present invention features methods of treating diseases that are caused by or that involve undesirable cell death. The delicate balance between cell growth and death allows for continuous remodeling and reshaping of cellular processes. When this balance is shifted, conditions that involve excess cell growth or cell death result.

In general, most diseases that are caused by excessive cell death are the result of the misregulation of apoptotic signaling proteins. Although our current understanding of apoptotic signaling pathways is far from complete, many of the key signaling proteins are known. For example, apoptosis can be triggered by extracellular toxins, calcium influx, lack of necessary growth factors, and activation of so called death receptors such as Fas and TNF-R. Downstream signaling proteins include Bcl-2 family members (Bcl-2, Bcl-Xₗ, Bax, Bad), proteins of the caspase/calpain family (activator caspases: 8, 9,10, 12 and effector caspases: 3, 6, 7), Apaf-1, as well as several transcription factors such as p53 and c-jun. Any disease in which signaling proteins are deregulated such that they shift the balance in favor of increased apoptosis, is treatable by the methods provided herein.

As the soluble form of CPG15 was first shown to promote cell survival in hippocampal and cortical neurons, it is a preferred embodiment of the invention that the methods or compositions comprising s-CPG15 be used to treat neurological conditions. s-CPG15 is useful in promoting the development, maintenance, or survival of neurons in vitro and in vivo, including central (brain and spinal chord), peripheral (sympathetic, parasympathetic, sensory, and enteric neurons), and motor neurons. Specific examples of neurological conditions include, but are not limited to, AD, PD, HD, stroke, ALS, peripheral neuropathies, trauma, and other conditions characterized by necrosis or loss of neurons. In addition, peripheral neuropathies associated with certain conditions, such as diabetes, AIDS, or chemotherapy can also be treated using the methods and compositions of the present invention.

Additional examples of neurological conditions considered treatable by the methods of the present invention include dementia with Lewy bodies (DLB), multiple system atrophy (MSA), muscular dystrophy, progressive supranuclear palsy (PSP), corticobasal degeneration, rare extrapyramidal conditions, multisystemic neuronal degeneration, synucleinopathies characterized by neuronal or glial inclusions of synuclein, Bell's palsy, Pick's disease, Kennedy disease, age-related conditions such as senility, Meniere's disease, multiple sclerosis (MS), spinocerebellar ataxia type I, spinobulbar muscular atrophy, and Machado-Joseph disease. The methods and compositions of the present invention can also be used to treat conditions of the retina and optic nerve, characterized by an increase in cell death in the retinal cells, and preferably the retinal ganglion cells. Examples include retinal degeneration, retinitis pigmentosa, diabetic retinopathy, age-related macular degeneration and general macular dystrophies.

The methods of the present invention can also be used to treat non-neuronal conditions which are caused by an increase in cell death. Examples of additional conditions characterized by an increase in cell death include liver disease; pulmonary disease; conditions of the skin such as trauma or bum; conditions of the bone, muscle joint, or cartilage; cardiovascular diseases and conditions including, cardiac ischemia, congestive heart disease, and myocardial infarction; autoimmune diseases such as rheumatoid arthritis; and immunodeficiency conditions such as those involving enhanced lymphocyte apoptosis.

Treatment of Conditions Involving Undesirable Cell Survival

The present invention features methods of treating conditions that involve undesirable cell survival. These conditions can result from a deregulation of signaling proteins such that they shift the balance towards a decrease or inhibition of cell death. Examples of such diseases include any form of cancer in which cell growth is left unchecked, any type of undesirable or tumor-associated angiogenesis, and conditions resulting from hyperactivity of the immune system.

Soluble CPG15 functions both to promote cell survival by preventing or inhibiting cell death and to promote cell growth and differentiation in specific cell types. As the promotion of differentiation typically results in a cessation of cellular proliferation, s-CPG15 itself can be used in certain cell types to inhibit proliferation of the cell. Purified forms of s-CPG15, as described above, can be used to treat diseases that involve undesirable cell survival such as cancer, undesirable or tumor-associated angiogenesis, and conditions resulting from hyperactivity of the immune system.

Alternatively, as the soluble form of CPG15 can prevent or inhibit cell death, inhibitors of s-CPG15 can also be used to promote or increase cell death. Examples of inhibitors of s-CPG15 include antisense nucleobase oligomers directed to s-CPG15, RNAi molecules directed to s-CPG15, antibodies that specifically recognize s-CPG15, and truncated or other dominant negative forms of CPG15 that can block the activity of s-CPG15.

Examples of cancers that can be treated by the methods and compositions of the present invention include bladder, blood, bone, brain, breast, cartilage, colon kidney, liver, lung, lymph node, nervous tissue, ovary, pancreatic, prostate, skeletal muscle, skin, spinal cord, spleen, stomach, testes, thymus, thyroid, trachea, urogenital tract, ureter, urethra, uterus, or vaginal cancer.

Antisense nucleobase oligomers

The present invention features the use of antisense nucleobase oligomers to downregulate expression of cpg15 mRNA which will lead to a reduction in expression of the soluble form of CPG15. By binding to the complementary nucleic acid sequence (the sense or coding strand), antisense nucleobase oligomers are able to inhibit protein expression presumably through the enzymatic cleavage of the RNA strand by RNAse H. Preferably the antisense nucleobase oligomer is capable of reducing s-CPG15, analogs, or fragments thereof, protein expression in a cell by at least 10% relative to cells treated with a control oligomer, more preferably 25%, and most preferably 50% or greater. Methods for selecting and preparing antisense nucleobase oligomers are well known in the art. Methods for assaying levels of protein expression are also well known in the art and include western blotting, immunoprecipitation, and ELISA.

RNA Interference

The present invention also features the use of RNA interference (RNAi) to inhibit expression of cpg15 which will lead to a reduction in the expression of s-CPG15. RNA interference (RNAi) is a recently discovered mechanism of post-transcriptional gene silencing (PTGS) in which double-stranded RNA (dsRNA) corresponding to a gene or mRNA of interest is introduced into an organism resulting in the degradation of the corresponding mRNA. In the RNAi reaction, both the sense and anti-sense strands of a dsRNA molecule are processed into small RNA fragments or segments ranging in length from 19 to 25 nucleotides (nt), preferably 21 to 23 nt, and having 2-nucleotide 3′ tails. siRNAs can also include short hairpin RNAs (shRNAs) in which both strands of an siRNA duplex are included within a single RNA molecule. Alternatively, synthetic dsRNAs, which are 19 to 25 nt in length, preferably 21 to 23 nt, and have 2-nucleotide 3′ tails, can be synthesized, purified and used in the reaction. These dsRNAs are known as “guide RNAs” or “short interfering RNAs” (siRNAs).

The siRNA duplexes then bind to a nuclease complex composed of proteins that target and destroy endogenous mRNAs having homology to the siRNA within the complex. Although the identity of the proteins within the complex remains unclear, the function of the complex is to target the homologous mRNA molecule through base pairing interactions between one of the siRNA strands and the endogenous mRNA. The mRNA is then cleaved approximately 12 nt from the 3′ terminus of the siRNA and degraded. In this manner, specific mRNAs can be targeted and degraded, thereby resulting in a loss of protein expression from the targeted mRNA.

The specific requirements and modifications of dsRNA are described in PCT Publication No. WO01/75164 (incorporated herein by reference). While dsRNA molecules can vary in length, it is preferable to use siRNA molecules which are 19- to 25- nt in length, most preferably 21- to 23- nucleotides in length, and which have characteristic 2- to 3- nucleotide 3′ overhanging ends typically either (2′-deoxy)thymidine or uracil. The siRNAs typically comprise a 3′ hydroxyl group. Single stranded siRNA as well as blunt ended forms of dsRNA can also be used. In order to further enhance the stability of the RNA, the 3′ overhangs can be stabilized against degradation. In one such embodiment, the RNA is stabilized by including purine nucleotides, such as adenosine or guanosine. Alternatively, substitution of pyrimidine nucleotides by modified analogs, e.g.,substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA can be prepared using any of the methods set forth in PCT Publication No. WO01/75164 or using standard procedures for in vitro transcription of RNA and dsRNA annealing procedures as described in Elbashir et al. (Genes & Dev., 15:188-200, 2001). Elbashir et al., supra, describe the preparation of siRNAs by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21 to 23 nt RNAs and the RNAs can then be eluted from the gel slices. In addition, chromatography (e.g., size exclusion chromatography), glycerol gradient centrifugation, and affinity purification with antibody can be used to isolate the 21 to 23 nt RNAs.

In the present invention, the dsRNA, or siRNA, is complementary to the mRNA sequence of a cpg15 mRNA and can reduce or inhibit expression of soluble CPG15. Preferably, the decrease in s-CPG15 protein expression is at least 10% relative to cells treated with a control dsRNA, shRNA, or siRNA, more preferably 25%, and most preferably at least 50%. Methods for assaying levels of protein expression are also well known in the art and include western blotting, immunoprecipitation, and ELISA. In one example, the siRNA is a small hairpin RNA having the following sequence: GGGCTTTTCAGACTGTTTG (SEQ ID NO: 2). In another example, the siRNA is a small hairpin RNA having the following sequence: GTTGAACGGCAGATATATT (SEQ ID NO: 3).

In the present invention, the nucleic acids used include any modification that enhances the stability or function of the nucleic acid in any way. Examples include modifications to the phosphate backbone, the intemucleotide linkage, or to the sugar moiety.

Antibodies

Antibodies that specifically bind to s-CPG15 can also be used to inhibit s-CPG15 biological activity and therefore to promote cell death. Such antibodies can be monoclonal or polyclonal and can include affinity-purified forms. When used in vivo for the treatment or prevention of conditions resulting from undesirable cell survival, the antibodies of the subject invention are administered to the subject in therapeutically effective amounts. Preferably, the antibodies are administered parenterally, intravenously by continuous infusion, intraventricularly in the brain, or intraocularly. The dose and dosage regimen depends upon the severity of the disease, and the overall health of the subject. The amount of antibody administered is typically in the range of about 0.01 to about 10 mg/kg of subject weight.

For parenteral administration, the antibodies are formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The antibodies typically are formulated in such vehicles at concentrations of about 1 mg/ml to 10 mg/ml.

Inhibitory Forms of CPG15

Dominant negative or truncated forms of CPG15 that can inhibit the biological activity of s-CPG15 can also be used to inhibit s-CPG15 biological activity and therefore to promote cell death. One example is t-CPG15, which lacks the amino acids encoding the GPI linkage sequence (i.e., t-CPG15 includes only amino acids 1-114 or 1-115 of the human CPG15 protein). In general, t-CPG15 is expressed from an engineered construct containing the nucleic acid sequence encoding CPG15 but lacking the nucleotides that encode the GPI linkage sequence. This truncated form of CPG15 does not follow the GPI linkage pathway but is instead secreted directly out of the cell without membrane attachment or modifications associated with membrane attachment.

A truncated form of CPG15 was described in Nedivi et al. (Science 281:1863-1866, 1998) and shown to inhibit dendritic arbor growth. At the time it was believed that this form of CPG15 was simply a non-functional form. However, we have discovered the soluble form of CPG15 to be the biologically active form, and we now believe that this truncated form acts as a dominant negative to inhibit the promotion of cell growth and survival by s-CPG15. The ability of the truncated form of CPG15 to inhibit the growth promoting and survival activities of the soluble CPG15 renders it a useful reagent for the treatment of conditions of excessive cell proliferation.

Dosages and Therapeutic Uses

By “therapeutically effective dose” herein is meant a dose that produces the therapeutic effects for which it is administered. The exact dose will depend on the condition to be treated, and may be ascertained by one skilled in the art using known techniques. In general, the s-CPG15 is administered at about 0.01 μg/kg to about 50 mg/kg per day, preferably 0.01 mg/kg to about 30 mg/kg per day, most preferably 0.1 mg/kg to about 20 mg/kg per day. The s-CPG15 may be given daily (e.g., once, twice, three times, or four times daily) or less frequently (e.g., once every other day, once or twice weekly, or monthly). In addition, as is known in the art, adjustments for age as well as the body weight, general health, sex, diet, time of administration, drug interaction, and the severity of the disease may be necessary, and will be ascertainable with routine experimentation by those skilled in the art.

A “subject” for the purposes of the present invention includes humans and other animals, preferably warm-blooded mammals including mice, rats, guinea pigs, hamsters, rabbits, cats, dogs, goats, sheep, cows, or monkeys. Thus, the methods are applicable to both human therapy and veterinary applications.

s-CPG15 can be administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, vaginally, rectally, intraarterially, intralesionally, intraventricularly in the brain, or intraocularly. The s-CPG15 can be administered continuously by infusion into the fluid reservoirs of the CNS, although bolus injection is acceptable, using techniques well known in the art, such as pumps or implantation.

Administration can be accomplished by a constant- or programmable-flow implantable pump or by periodic injections. Sustained release systems can also be used. Generally, where the condition permits, one should formulate and dose the s-CPG15 for site-specific delivery. Administration can be continuous or periodic.

Semipermeable, implantable membrane devices are useful as a means for delivering drugs in certain circumstances. For example, cells that secrete s-CPG15 can be encapsulated, and such devices can be implanted into a subject, for example, into the brain or spinal cord (CSF) of a subject suffering from Parkinson's Disease. See, U.S. Pat. Nos. 6,042,579; 4,892,538; 5,011,472; 5,106,627; PCT Applications WO 91/10425; 91/10470; Winn et al., (Exper. Neurology, 113:322-329, 1991); Aebischer et al., (Exper. Neurology, 111:269-275, 1991); and Tresco et al., (ASAIO, 38:17-23, 1992), each of which is herein incorporated by reference. The pharmaceutical compositions of the present invention comprise s-CPG15 in a form suitable for administration to a subject. In the preferred embodiment, the pharmaceutical compositions are in a water soluble form, and may include such physiologically acceptable materials as carriers, excipients, stabilizers, buffers, salts, antioxidants, hydrophilic polymers, amino acids, carbohydrates, ionic or nonionic surfactants, and polyethylene or propylene glycol. The s-CPG15 may be in a time-release form for implantation, or may be entrapped in microcapsules using techniques well known in the art. Additional excipients useful for pharmaceutical compositions include any of those listed in U.S. patent application Ser. No. 20030176672, herein incorporated by reference.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

The compositions hereof including lyophilized forms, are prepared in general by compounding the components using generally available pharmaceutical compounding techniques, known per se. Methods well known in the art for making formulations are found, for example, in “Remington: The Science and Practice of Pharmacy” (20^{th} ed., ed. A. R. Gennaro, 2000, Lippincott Williams & Wilkins, Philadelphia, Pa.). A particular method for preparing a pharmaceutical composition of s-CPG15 hereof comprises employing purified (according to any standard protein purification scheme) s-CPG15, in any one of several known buffer exchange methods, such as gel filtration or dialysis.

The s-CPG15 can also be delivered via a nucleic acid encoding s-cpg15. The nucleic acid can be any nucleic acid (DNA or RNA) including genomic DNA, cDNA, and mRNA encoding any soluble form of cpg15 shown to promote cell survival or reduce or prevent cell death. The nucleic acids of the present invention include any modification that enhances the stability or function of the nucleic acid in any way. Examples include modifications to the phosphate backbone, the internucleotide linkage, or to the sugar moiety.

To simplify the manipulation and handling of the nucleic acid encoding cpg15, the nucleic acid is preferably inserted into a cassette where it is operably liked to a promoter. The promoter must be capable of driving expression of s-cpg15 in the desired target cell. Selection of the appropriate promoter and generation of the recombinant s-cpg15 expressing vector are techniques well known to one skilled in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome mediated transfection, electroporation, calcium phosphate/DEAE dextran methods, gene gun, and microinjection.

Gene delivery using viral vectors such as adenoviral, retroviral, lentiviral, or adeno-asociated viral vectors can also be used. Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller and Rosman, Biotechniques 7:980-990, 1989; Rosenberg et al., N. Engl. J Med 323:370, 1990, Groves et al., Nature, 362:453-457, 1993; Horrelou et al., Neuron, 5:393-402, 1990; Jiao et al., Nature 362:450-453, 1993; Davidson et al., Nature Genetics 3:2219-2223, 1993; Rubinson et al., Nature Genetics 33, 401-406, 2003; U.S Pat. Nos. 6,180,613; 6,410,010; 5,399,346 all hereby incorporated by reference). These vectors include adenoviral vectors and adeno-associated virus-derived vectors, retroviral vectors (e.g., Moloney Murine Leukemia virus based vectors, Spleen Necrosis Virus based vectors, Friend Murine Leukemia based vectors, lentivirus based vectors (Lois C. et al., Science, 295:868-872, 2002; Rubinson et al., supra), papova virus based vectors (e.g., SV40 viral vectors), Herpes-Virus based vectors, viral vectors that contain or display the Vesicular Stomatitis Virus G-glycoprotein Spike, SemlikiForest virus based vectors, Hepadnavirus based vectors, and Baculovirus based vectors. Adenovirus, adeno-associated virus, and lentivirus are the preferred viral vectors for treatment of neurological conditions since they do not require recipient cells to be actively dividing.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of s-CPG15.

Any of the aforementioned delivery methods can also be used for delivery of antisense nucleobase oligomers, RNAi, dominant negative forms of CPG15, or antibodies directed to s-CPG15. The delivery of such s-CPG15 inhibitors as therapeutic agents can be useful for the treatment of diseases characterized by undesirable cell survival.

In Vitro Uses

s-CPG15 can be used in a variety of in vitro applications. These applications include adding s-CPG15 to cell culture media to promote the growth and survival of cells grown in culture. Purified s-CPG15 can also be used in stem cell growth applications where both the growth and survival promoting functions as well as the differentiating functions are useful. In addition, purified s-CPG15 can be used for applications relating to repairing and regenerating damaged tissue or organs by growing the tissue or organs ex vivo in the presence of s-CPG15. These examples are described in detail below.

Cell Culture

s-CPG15 is useful as a component of culture media for use in culturing cells in vitro or ex vivo. In one example the cells are cell of the nervous system. The s-CPG15 can be added to the media before or after the addition of media to the cells. s-CPG15 may also be added directly to the cells when needed or as a part of any other solution added to the cells. The amount of s-CPG15 added to the media is dependent on the type of cells used and the number of passages of the cells but can be determined empirically by the skilled artisan. Typically the amount of s-CPG15 added to the media will range from 0.001 μg/mL to 100 μg/mL, preferably 0.1 μg/mL to 100 μg/mL, and most preferably 1.0 μg/mL to 10 μg/mL.

Cell Growth and Differentiation

Differentiation of stem cells can be accomplished by exposing the cells to s-CPG15. Stem cells can be proliferated in culture, and then differentiated in vitro or in situ into the cell types needed for therapy. Some of the progenitor or stem cell types which can be induced to differentiate using the purified s-CPG15 of the present invention include, embryonic stem cells, endothelial, muscle, neural, pancreatic, hepatocyte, chondrocyte, cardiomyocyte, oligodendrocyte, and hematopoietic progenitor cells. Cells induced to differentiate into their specialized forms can then be used for therapeutic purposes. Stem cells induced to expand or differentiate in the presence of s-CPG15 can be used for transplantation (e.g, organ, tissue, or bone marrow cell, or more specialized forms of transplantation where, for example, chondrocytes can be implanted into a joint surface defect in need of repair); the treatment of insulin dependent diabetes; the treatment of hematopoeitc conditions resulting from the loss of platelets or other hematopoietic cells; the treatment of cardiac injuries or liver injuries; and the treatment of neurodegenerative conditions such as epilepsy, stroke, ischemia, Huntington's disease, Parkinson's disease and Alzheimer's disease. Stem cells induced to differentiate using s-CPG15 may also be appropriate for blood vessel repair or replacement. Stem cells induced to differentiate using s-CPG15 may also be appropriate for treating demyelinating conditions, such as Pelizaeus-Merzbacher disease, multiple sclerosis, leukodystrophies, neuritis and neuropathies. In one example, stem cells that have been induced to differentiate along a neuronal or myogenic lineage can be transplanted into the affected regions of a subject in need of cell replacement therapy.

Purified s-CPG15 can be added to the media used to culture the growing progenitor or stem cells. The s-CPG15 can be added to the media before or after the addition of media to the cells. s-CPG15 may also be added directly to the cells when needed or as a part of any other solution added to the cells. The amount of s-CPG15 added to the media is dependent on the type of cells used and the number of passages of the cells but can be determined empirically by the skilled artisan. Typically the amount of s-CPG15 added to the media will range from 0.001 μg/mL to 100 μg/mL.

Tissue or Organ Transplantation

Purified s-CPG15 can also be used to promote cell survival and/or differentiation for tissue and organ transplantation, the repair of diseased or damaged tissues and organs, and replacement tissue and organ engineering. The survival and differentiation promoting functions of s-CPG15 make this protein amenable as an added nutrient or type of growth factor in methods for sustaining organ or tissue survival in culture, e.g., prior to transplantation of the organ or tissue.

Desirably, the organ is a bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, blood vessel, skin, bone, or cartilage, or any part thereof of these organs. In desired embodiments, the tissue includes one or more cell-types derived from bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, or cartilage.

In addition, purified s-CPG15 can be used to promote growth and differentiation in applications involving the growth of natural or synthetic tissues or organs in vitro. Tissue engineering is a method by which new living tissues are created in the laboratory to replace diseased or traumatized tissue. Methods for expanding various cell types used in tissue engineering are described in U.S. Pat. No. 6,582,960, herein incorporated by reference). In one example, s-CPG15 is added to a matrix or scaffold used for the growth of neuronal cells for central nervous system nerve regeneration. s-CPG15 can be perfused into any in vitro system used for the survival or promotion of tissue or organ growth. Administration of s-CPG15 can also be continued after the organ or tissue has been transplanted into the subject.

One particular strategy that has been created to regenerate new tissue is to (i) isolate specific cells from tissue; (ii) expand the isolated cells in vitro; and (iii) implant the expanded cells into the diseased or traumatized tissue so that the implanted cells proliferate in vivo and eventually replace or repair the tissue defect (Langer et al. Science 260:920-926, 1993). This technique has been applied to a variety of cell types and tissue defects (for example see Brittberg et al., N. Engl. J Med., 331:889-895, 1994; Rheinwald et al., Cell, 6:331-344, 1975; Langer et al., supra). Isolated cells can be either differentiated cells from specific tissues or undifferentiated progenitor cells (stem cells). In both cases, establishment of appropriate culture conditions for cell expansion using s-CPG15 is extremely important in order to maintain or improve their potential to regenerate structural and functional tissue equivalents (Rheinwald et al., supra).

According to the present invention, any cell type desirable for use in tissue engineering that can be isolated is used to regenerate tissue. Non-limiting examples include endothelial cells, muscle cells, skin cells, hepatocytes, chondrocytes, and melanocytes. Desirably, s-CPG15 is added in an amount sufficient to promote the expansion of the cells or the tissue while preserving the appropriate differentiation properties of the cells to ensure successful regeneration of high quality tissue or organ for implantation.

Assays for Cell Death

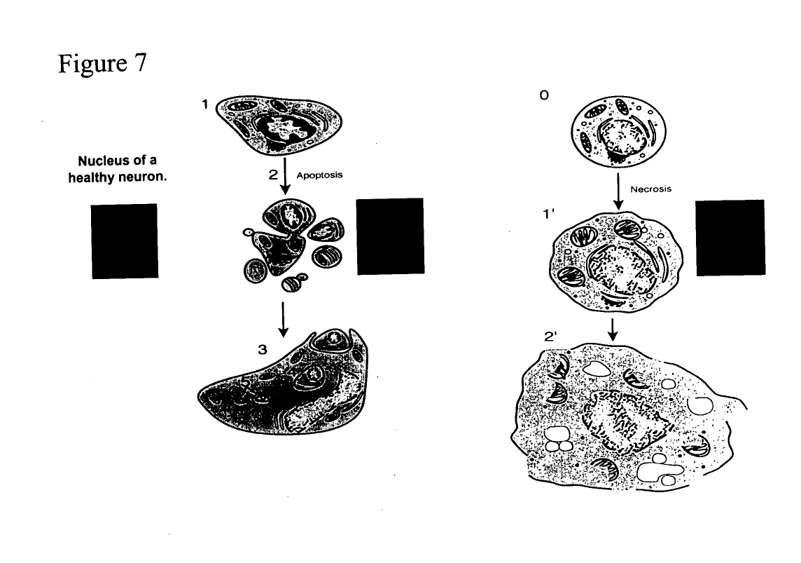

There are many assays for cell death that are known to the skilled artisan. The assays differ depending on the type of cell death being detected, and, in some cases, the cell types of interest. FIG. 7 illustrates some of the intracellular differences between a cell undergoing apoptosis and necrosis. In an apoptotic cell, the cell membrane-bound apoptotic bodes are engulfed and degraded by a macrophage. The nuclear chromatin becomes pyknotic and condenses against the nuclear membrane. In contrast, necrosis involves only modest condensation of chromatin. One general method for distinguishing between a healthy, apoptotic, or a necrotic cell is through the use of Hoechst 33342 staining of the chromatin.

Some specific examples of assays for apoptosis and necrotic cell death are provided below. These examples are meant to illustrate the invention. They are not meant to limit the invention in any way.

Assay for Necrotic Cell Death

Necrosis is a passive process in which collapse of internal homeostasis leads to cellular dissolution. The process involves loss of integrity of the plasma membrane and subsequent swelling, followed by lysis of the cell (Schwartz, et al., Proc. Natl. Acad. Sci. USA, 90:980-984, 1993). Propidium iodide (PI) is known to bind to the DNA of cells undergoing primary and secondary necrosis (Vitale, et al., Histochemistry, 100:223-229 1993). Necrotic cell death is characterized by loss of cell membrane integrity and permeability to dyes such as PI. Necrosis may be distinguished from apoptosis in that cell membranes remain intact in the early stages of apoptosis. As a consequence a dye exclusion assay using PI should be used in parallel with an assay for apoptosis, as described below in order to distinguish apoptotic from necrotic cell death, and the percentage of cells undergoing necrosis may be measured at various times before and after treatment with s-CPG15.

Assay for Apoptotic Cell Death

Detection of programmed cell death or apoptosis may be accomplished using standard methods known to those in the art. The percentage of cells undergoing apoptosis may be measured at various times before and after treatment with s-CPG15 and compared with a control population of cells not treated with s-CPG15. The morphology of cells undergoing apoptotic cell death is characterized by a shrinking of the cell cytoplasm and nucleus and condensation and fragmentation of the chromatin (Wyllie, et al., J Pathol. 142:67-77, 1984). One of the earliest events in programmed cell death is the translocation of phosphatidylserine, a membrane phospholipid from the inner side of the plasma membrane to the outer side. Annexin V is a calcium-dependent phospholipid binding protein that has a high affinity for membrane bound phosphatidylserine and thus annexin V-FITC can be used to stain cells undergoing apoptosis with detection and quantitation of apoptotic cells by flow cytometry or any other method of fluorescent detection (Vermes, et al., J Immunol. Methodol., 184:39-51, 1995; Walton, et al., Neuroreport, 3:3871-3875, 1997). Accordingly, annexin V, when attached to a solid support such as a bead or a resin, can be used as an affinity ligand for binding apoptotic cells in solution. Similarly, annexin V is the basis for a fluorescent-activated cell sorting (FACS) separation process, another assay method well-known to the skilled artisan.

Additional assays for apoptosis in neuronal cells are disclosed by: Melino et al., Mol. Cell. Biol. 14:6584-6596, 1994; Rosenbaum et al., Ann. Neurol. 36:864-870, 1994; Sato et al., J Neurobiol., 25:1227-1234, 1994; Ferrari et al., J. Neurosci., 1516:2857-2866, 1995; Talley et al., Mol. Cell Biol. 1585:2359-2366, 1995; and Walkinshaw et al., J Clin. Invest., 95:2458-2464, 1995; and U.S Pat. Nos. 6,174,869 and 6,379,882, each of which is herein incorporated by reference.

Quantitation of Cell Survival

Cell survival may be measured at various times before and after treatment with s-CPG15 using the MTT assay. The MTT assay is a measure of mitochondrial activity in cells and is a general indicator of cell viability. The MTT assay is based on the ability of living cells to take in and process the dye known as MTT (3-(4,5-dimethylthiazol-2-yl)2,5-diphenyl tetrazolium bromide; Sigma Chemical Co., St. Louis, Mo.), an active process which dead cells cannot complete. The assay is described in Mosmann et al. (J. Immunol. Meth. 65:55-63, 1983); Barres et al. (Cell 70:31-46, 1992); and Barres et al. (Development 118:283-295, 1993). MTT is added to the cell culture and incubated at 37° C. for one hour. Viable cells with active mitochondria cleave the tetrazolium ring into a visible dark blue formazan reaction product. Viable and dead cells are counted by bright field microscopy at various times, e.g., 24, 48, or 72 hours after treatment with s-CPG15.

Interpretation of Results

Cell death can be evaluated using light microscopy following the staining of cells with the mitochondrial dye MTT, or by fluorescent/light microscopy following the staining of cells with propidium iodide (PI) or annexin V. Cell death is also evaluated by FACS analysis following staining with PI or annexin V. The percentage of apoptotic cells may be determined based on the percentage of annexin V positive cells that are not PI or MTT positive. However, there are some cells in later stages of apoptosis that also exhibit a loss of cell membrane integrity and stain positive with PI (i.e., they are undergoing secondary necrosis).

Animal Models

The use of animals in medical research is a major way to increase our knowledge of the pathogenesis and alleviation of diseases in both animals and humans. Experiments on animals with induced diseases or conditions can be done under controlled conditions. Mechanisms relating to basic cellular processes such as cell division and apoptosis are highly conserved between species, particularly within mammals. A successful non-human animal model of neuronal cell death offers the prospect of understanding the origin and mechanisms of many neuronal conditions. Existing non-human animal models of neurological conditions can also be used to further explore therapies for neurological conditions. Non-human animals can include mice, rats, guinea pigs, hamsters, rabbits, cats, dogs, goats, sheep, cows, monkeys, or other mammals. Animal models can also be used to explore therapies for non-neuronal conditions.

Animal models can also include any transgenic animals, cpg15 gene knock out animals, or specific crosses of transgenic or knock out animals. For example, one animal model could be achieved by crossing a cpg15 transgenic animal with a separate animal model for a neurological condition. This type of cross could be very useful in determining the ability of s-CPG15 to rescue the defect causing the neurological condition.

Animals may be obtained from a variety of commercial sources, for example Charles River Laboratories, and housed under conditions of controlled environment and diet.

Screen for Interacting Molecules

We describe here the ability of s-CPG15 to block cell death. Although we do not wish to be bound to a particular theory, it is likely that s-CPG15 functions to inhibit one or more signaling proteins that induce cell death. As many of these cell death pathways are commonly used by many different types of cells, s-CPG15 can therefore be used as a screening tool to identify interacting proteins that are important for the induction of cell death pathways.

It is also possible that s-CPG15 functions as a survival factor and may interact with a receptor or protein that is required to initiate cell survival. By using s-CPG15 as a screening tool, it may be possible to identify a protein or receptor important for cell survival. Modulation of such a protein or receptor through the use of agonists or antagonists could then be used to initiate or inhibit cell death pathways.

There are many types of screens for interacting proteins known in the art, all of which are included herein as screens for s-CPG15 interacting proteins. Some examples include affinity chromatography using immobilized s-CPG15, co-immunoprecipitations, and genetic screens, such as yeast two-hybrid screens, and variations thereof. In one example, a fusion construct encoding s-CPG15 fused to alkaline phosphatase is used to detect binding to a tissue sample or a library (Flanagan J.G., Curr. Biol. 9:R469-470, 1999; Zhang et al., J. Neurosci. 16:7182-7192, 1996; Cheng et al., Cell 82:371-381, 1995; Cheng et al., Cell 79:157-168, 1994). This method can be used to identify tissues which contain potential s-CPG15 interacting proteins and which can then be used to generate expression libraries for additional screening. In another example, affinity chromatography using purified s-CPG15 is employed. In this approach the s-CPG15 is expressed, purified, and immobilized using any number of art-known methods including direct immobilization of s-CPG15 to any type of resin (e.g., sepharose or cellulose beads), immobilization through a protein tag on the s-CPG15 such as GST or His tag interacting with an appropriate resin (e.g., glutathione sepharose or agarose for the GST tag and nickel sepharose or agarose for the His tag), or immobilization through an interaction with an anti-CPG15 antibody which is linked to beads or a resin. Immobilization of the purified s-CPG15 is preferably done under conditions that allow proteins associated with the s-CPG15 to remain associated with it. Such conditions may include the use of buffers that minimize interference with protein-protein interactions.

A test mixture is then mixed with the immobilized s-CPG15. The test mixture can be a cell lysate from any type of mammalian cell culture. Preferred cell types include 293, 293T, PC12 cells, HeLa, BHK, 3T3, HaK, or primary neuronal cells. In addition, tissue samples such as brain tissue samples can also be homogenized and used in the screen. The cell/tissue lysate can be unlabeled or radioactively labeled in order to easily identify interacting proteins. Any interacting proteins will be immobilized onto the s-CPG15 resin and the beads are then washed several times to remove any non-specific binding proteins. After washing, the s-CPG15 bound to the beads and any interacting proteins are incubated under denaturing conditions to release the proteins and the proteins are then separated by electrophoresis. Various types of protein gels can be used including sodium dodecyl sulfate-polyacrylamide gel electrophoresis (SDS-PAGE) followed by autoradiography for labeled cell lysates or SDS-PAGE followed by Coomassie blue staining or silver staining for unlabeled lysates. These protein-staining methods are standard, art-known techniques. Potential interacting proteins are purified and sequenced. Compounds isolated by this method (or any other appropriate method) may, if desired, be further purified (e.g., by high performance liquid chromatography). Compounds that are identified as binding to a polypeptide of the invention with an affinity constant less than or equal to 10 mM are considered particularly useful in the invention.

Proteins that are identified as interacting proteins using any type of screen described above are then assayed for their ability to promote or inhibit apoptosis as measured by any standard assay such as those described herein, and can be used accordingly. For example, it is likely that s-CPG15 interacts with pro-apoptotic proteins to inhibit their function. Any pro-apoptotic proteins identified in this screen can then be used therapeutically to treat diseases which are a result of a decrease in or inhibition of apoptosis. The best example of such diseases includes any form of cancer in which cellular proliferation is uncontrolled. Treatment of cancer cells with any pro-apoptotic protein identified in this screen could induce apoptosis thereby reducing the proliferative capacity of these cells.

Screening Assays for Compounds that Modulate s-CPG15

As discussed above, we have discovered that the expression of s-CPG15 is useful in promoting cell survival and cell differentiation. Based on these discoveries, compositions of the invention are useful for the high-throughput low-cost screening of candidate compounds to identify those that modulate the expression of an s-CPG15 protein or nucleic acid for therapeutic purposes.

Any number of methods are available for carrying out screening assays to identify new candidate compounds that alter the expression of an s-CPG15 nucleic acid molecule. In one working example, candidate compounds are added at varying concentrations to the culture medium of cultured cells expressing an s-CPG15 nucleic acid molecule. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate compound. A compound that promotes an alteration such as an increase in the expression of an s-CPG15 gene, nucleic acid molecule, or protein or a functional equivalent thereof, is considered useful in the invention; such a molecule may be used, for example, as a therapeutic to treat conditions.

In another working example, the effect of candidate compounds may be measured at the level of protein expression using the same general approach and standard immunological techniques, such as Western blotting or immunoprecipitation with an antibody specific for a s-CPG15 protein. For example, immunoassays may be used to detect or monitor the expression s-CPG15 in an organism or a cell line. Polyclonal or monoclonal antibodies (produced as described herein) may be used in any standard immunoassay format (e.g., ELISA, western blot, and immunoprecipitation) to measure the level of the protein. In some embodiments, a compound that promotes an alteration such as an increase in the expression or biological activity of s-CPG15 protein is considered particularly useful. Again, such a molecule may be used, for example, as a therapeutic to for conditions of excessive cell death.

The aforementioned growth and differentiation assays, as well as the apoptotic cell death assays, such as the hippocampal cell starvation assay described in Example 5, are also useful for assessing the ability of compounds (for example, organic compounds; small molecules; nucleic acid ligands such as DNA, RNA, or mixed nucleotide aptamers; ligands; synthetic chemicals; proteins; agonists; and antagonists) to modulate the ability of s-CPG15 to rescue cells from apoptosis or to promote cell survival and differentiation. The method of screening may also involve high-throughput techniques employing standard computerized robotic and microtiter plates as is described below.

In one example, the method involves screening a library for therapeutically-active agents by employing, for example, the hippocampal starvation assays described herein. Based on our demonstration that s-CPG15 can prevent starvation-induced apoptosis, it will be readily understood that an agent which enhances the ability of s-CPG15 to prevent starvation-induced apoptosis could be used as an effective therapeutic agent in a subject suffering from a disease associated with inappropriate cell death.

Accordingly, the methods of the invention simplify the evaluation, identification, and development of active agents such as drugs for the treatment of conditions caused by excessive cell death.

In general, the chemical screening methods of the invention provide a straightforward means for selecting natural product extracts or agents of interest from a large population which are further evaluated and condensed to a few active and selective materials. Constituents of this pool are then purified and evaluated in the methods of the invention to determine their ability to modulate the cell-survival promoting activity of sCPG15.

Test Extracts and Agents

In general, novel drugs are identified from large libraries of both natural product or synthetic (or semi-synthetic) extracts or chemical libraries according to methods known in the art. The screening methods of the present invention are appropriate and useful for testing agents from a variety of sources for possible activity in vitro. The initial screens may be performed using a diverse library of agents, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

Virtually any number of chemical extracts or compounds known to those skilled in the art of drug discovery and development can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds, including nucleic-acid ligands such as apatmers. Synthetic compound libraries are commercially available from Nanoscale Combinatorial Synthesis Inc., Mountain View, Calif., ChemDiv Inc., San Diego, Calif., Pharmacopeia Drug Discovery, Princeton, N.J., and ArQule Inc., Medford, Mass. Alternatively, libraries of natural compounds in the form of bacterial, fungal, plant, and animal extracts are commercially available from a number of sources, including Phytera Inc., Worcester, Mass. and Panlabs Inc., Bothell, Wash. In addition, natural and synthetically produced libraries are produced, if desired, according to methods known in the art, e.g., by standard extraction and fractionation methods. Devices for the preparation of combinatorial libraries are also commercially available, for example, Advanced ChemTech, Louisville, K.Y. and Argonaut Technologies Inc., San Carlos, Calif. Furthermore, if desired, any library or compound is readily modified using standard chemical, physical, or biochemical methods.

When a crude extract is found to have activity that modulates s-CPG15 cell survival promoting activity in vitro, further fractionation of the positive lead extract is necessary to isolate chemical constituents responsible for the observed effect. Thus, the goal of the extraction, fractionation, and purification process is the careful characterization and identification of a chemical entity within the crude extract having activity that modulates the ability of s-CPG15 to promote cell survival. Methods of fractionation and purification of such heterogenous extracts are known in the art

Since many of the compounds that constitute currently available combinatorial and natural products libraries, as well as those found in natural products preparations, are not characterized, the screening methods of this invention provide novel compounds which are active as agonists or antagonists in the particular assays, in addition to identifying known compounds which are active in the screens. Therefore, this invention includes such novel compounds, as well as the use of both novel and known compounds in pharmaceutical compositions and methods of treating disease characterized by excessive cell death such as AD, PD, HD, and ALS.

### EXAMPLES

The features and other details of the invention will now be more particularly described and pointed out in the following examples describing preferred techniques and experimental results. These examples are provided for the purpose of illustrating the invention and should not be construed as limiting.

### Example 1

### Expression of cpg15 mRNA in Various Tissues

To assess the expression pattern of cpg15 RNA, RT-PCR was performed on RNA from various tissues including brain, eye, heart, lung, liver, kidney, spleen, intestine, muscle, and testis. For each reaction, cDNA synthesized from 125 ng of total RNA was amplified for 24 cycles (cpg15) or 19 cycles (actin) using specific primers. cpg15 is expressed in brain and liver, and at lower levels in eye, heart, lung, and spleen, suggesting it may have a role in promoting cell survival and differentiation in these tissues.

### Example 2

### Expression of cpg15 mRNA in Developing Rat Brains

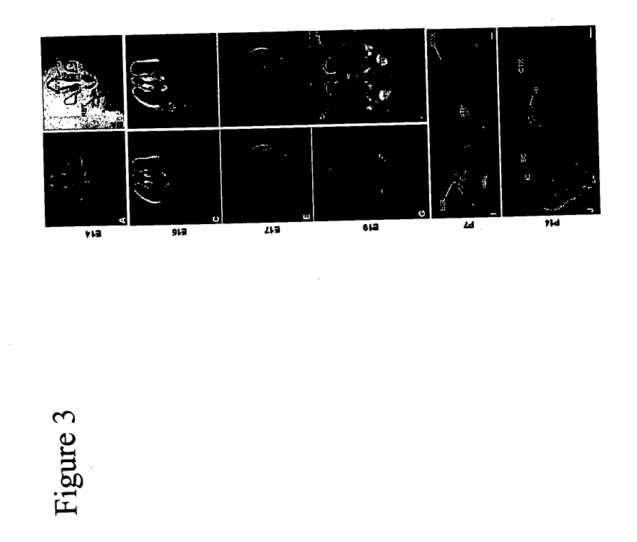

cpg15 was identified in a screen for activity-regulated genes involved in synaptic plasticity. Consistent with its isolation as an activity-regulated gene, late cpg15 expression corresponds with the timing of critical periods for activity-dependent plasticity and is dependent on action potential activity. However, cpg15 is also expressed in an activity-independent manner at earlier stages of brain development, suggesting that in addition to its previously characterized role in activity-dependent aspects of circuit formation and maturation, cpg15 may play another role early in development. To investigate this possibility we examined early cpg15 expression by performing in situ hybridizations on sections from embryonic rat brains. At the earliest times tested, on embryonic days 14 (E14) and 15 (E15), cpg15 mRNA is present in the cortical plate, in the ventricular zone of the dorsal thalamus, and in retinal ganglion cells (FIGS. 3A-3D), coinciding with rapid neuronal proliferation and apoptosis in these regions. At E17-E19 cpg15 is expressed in the telencephalic and dorsal diencephalic subventricular zones, in the hippocampal primordia, and at postnatal day 7 (P7) can be see in the external granular layer of the cerebellum (FIG. 3E-3I). Thus, in all these regions early cpg15 expression is temporally correlated with expansion of the progenitor pool and the apoptotic elimination of superfluous neuroblasts required for correct brain morphogenesis. Interestingly, cpg15 is not expressed in all the proliferative zones, and is markedly absent from the olfactory epithelium and ganglionic eminence (FIGS. 3A-3D, 3G-3H), suggesting that it may be specific for certain cell types. From E19 to P7 cpg15 mRNA can also be seen in the trigeminal ganglia, sensory thalamus, and various brain stem nuclei (FIG. 3G-3I), at times of afferent ingrowth, target selection and synaptogenesis in these structures. Thus, cpg15 is also expressed when target-derived trophic support is crucial for protection against apoptosis used to match neuron number with target size. From P0, cpg15 expression in the cerebral cortex is down-regulated to undetectable levels, concurring with cessation of the period for apoptosis in this region (FIG. 3I). At P 14, cpg15 mRNA re-appears, not in the ventricular or subventricular zones, but rather in the differentiated cortical layers where activity-dependent plasticity is thought to occur postnatally. cpg15 MRNA patterns are thus consistent with a role as a survival factor during early brain morphogenesis, and a late role in the structural remodeling and synaptic maturation associated with developmental and adult plasticity.

### Example 3

### Soluble CPG15 transfers to non-transfected cells

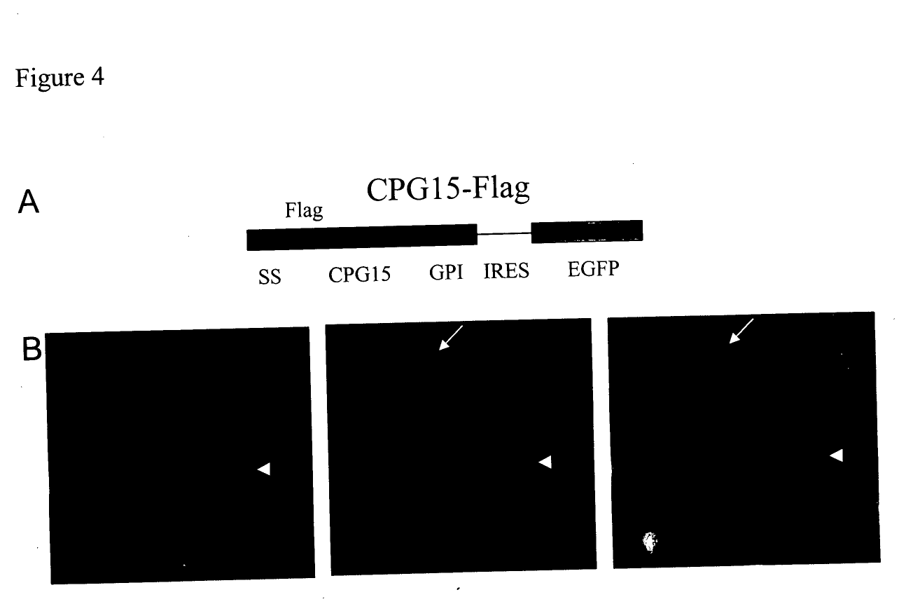

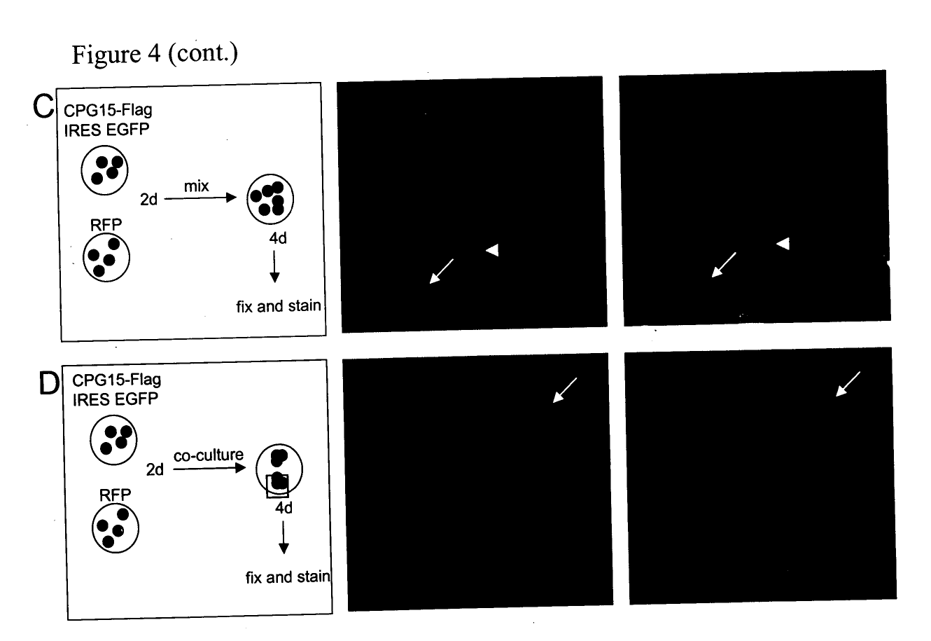

The full-length cpg15 was epitope tagged by insertion of the FLAG peptide between the secretion signal and the cpg15 core domain, then cloned into an IRES-EGFP vector (FIG. 4A). This vector expressed CPG15-FLAG and EGFP independently, therefore the green EGFP signal was used as a separate marker for transfected cells. 293T cells were transfected with CPG15-FLAG IRES EGFP and stained with Rhodamine (red) for the detection of CPG15-FLAG. Transfected cells expressing EGFP show a green signal (FIG. 4B). Immunohistochemistry with an anti-FLAG monoclonal antibody revealed a staining pattern indicative of membrane localization (FIGS. 4C and 4D), consistent with CPG15's GPI link to the cell surface. Unexpectedly, CPG15 was also detected on untransfected EGFP-negative cells, suggesting intercellular transfer of CPG15 from transfected cells to their untransfected neighbors. As indicated by the arrow, cells that were not transfected (not expressing EGFP) did show a positive staining signal for CPG15-FLAG indicating that the soluble CPG15-FLAG transferred to non-transfected cells.

In FIG. 4C, 293T cells were transfected separately with CPG15-FLAG IRES EGFP or a plasmid expressing red fluorescent protein (RFP). Two days post-transfection, cells were mixed and stained for CPG15 using Cy5 (shown in blue) after four days. Cells expressing RFP were definitely not transfected with the CPG15-FLAG expressing plasmid, yet these cells did show CPG15-FLAG staining as indicated by the arrows. In FIG. 4D, 293T cells were transfected separately with CPG15-FLAG IRES EGFP or a plasmid expressing RFP. Two days post-transfection, the cells were co-cultured on the same coverslip without contact and then stained for CPG15-FLAG four days later using Cy5. CPG15-FLAG staining was detectable in cells not transfected and not in contact with transfected cells. These results suggest the existence of a soluble form of CPG15 that can be transferred between isolated cells.

### Example 4

### Soluble CPG15 Exists as a Separate Extracellular Form

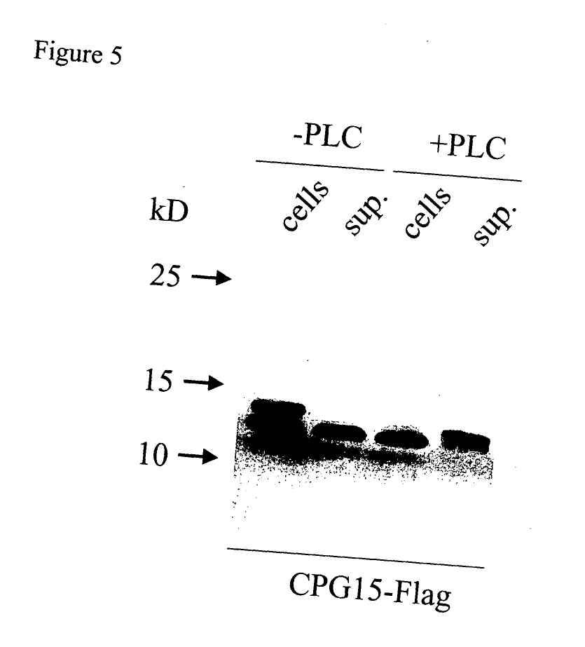

To verify the presence of a soluble form of CPG15 in supernatants from CPG15transfected cells, we prepared Western blots from these cells containing proteins from whole cell extracts as well as their supernatants (FIG. 5). When probed with the anti-FLAG antibody, two CPG15 bands with distinct molecular weights were observed in the lane containing protein from cell extracts. The lower molecular weight band was also present in the supernatant, suggesting that this may be a soluble form of CPG15. To test whether the higher molecular weight band represents CPG15 in its membrane bound GPI-linked form, CPG15-FLAG transfected cells were treated with phospholipase C (PLC). PLC cleaves GPI-anchors causing the release of GPI-anchored proteins from the cell-surface (Hooper, Proteomics 1:748-755, 2001). PLC treatment resulted in elimination of the larger molecular weight protein from the whole cell extract and its concurrent appearance in the supernatant (FIG. 5). In contrast, the presence of the lower molecular weight protein remained unaltered with PLC treatment.

To confirm that CPG15 is expressed in both a membrane-bound and soluble form in vivo, membrane and soluble fractions were prepared from the brains of E14, E18, and adult rats, and were probed on Western blots with an antibody against CPG15 (Nedivi et al., Science 281:1863-1866, 1998). Membrane and soluble fractions from brains were prepared as described (Kim et al., Neuron 20:683-691, 1998) with the following modifications. Briefly, brains from E14 and E18, and cortices from adult Sprague-Dawley rat were homogenized using a Teflon homogenizer in 5 ml ice-cold buffer (320 mM sucrose, 10 mM Tris-HCl, pH7.4, 5 mM EDTA, 1:100 Protease Inhibitor Cocktail (Sigma)) per 1 g of tissue. The homogenates were centrifuged at 700 ×g for 10 minutes at 4° C., followed by centrifugation of the supernatant at 28,000 ×g for 16 minutes at 4° C.

The high-speed pellet (P2) was resuspended in 500 μl ice-cold TE buffer (10 mM TrisHCl, 5 mM EDTA, pH 7.4). Protein concentration of the pellet (P2) and supernatant were determined using the Bradford assay (Gibco). Samples containing 20-40 μg protein were diluted 1:1 in loading buffer (Laemmli, Nature 227:680-685, 1970), boiled for 5 minutes, then separated on 16.5% Tris-Tricine gels (Schagger et al., Analytical Biochemistry 166:368-379, 1987). Western blots were incubated with a polyclonal anti-CPG15 antibody (Nedivi et al, supra). Clean fractionation of soluble versus membrane compartments was confirmed by staining with a monoclonal antibody specific for the membrane associated protein transferrin-receptor (1:1000, Zymed), followed by a goat anti-mouse HRP secondary antibody (1:5000, Sigma), and a polyclonal antibody specific for the soluble protein Akt (1:1000, Cell Signaling Technology) followed by a goat anti-rabbit HRP secondary antibody (1:2500, Jackson Immuno Research). Prior to antibody staining the blot was incubated for two minutes with Ponceau S solution (Sigma) to determine protein content as a loading control.

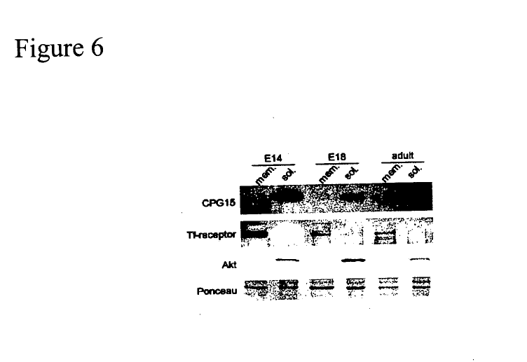

CPG15 could be detected predominantly in the soluble fraction, with low levels of the membrane-bound form present only in the adult (FIG. 6). These in vivo results concur with the in vitro data demonstrating that CPG15 exists in a soluble as well as a membrane-bound GPI-linked form. Yet, in vivo the prevalent form of CPG15 is soluble, especially at early developmental times. These data support the hypothesis that soluble CPG15 may be the primary mediator of CPG15's role as a survival factor, particularly during embryonic brain development, and perhaps of its later role as an activity-regulated growth and differentiation factor.

### Example 5

### Starvation and Apoptosis Assay Protocols

As shown in FIG. 8, primary hippocampal or cortical neurons were plated essentially as described in Zhou et al. (FEBS Letters 526:21-25, 2002). Briefly, E18 Sprague-Dawley rat embryos were collected in ice-cold Hank's buffered salt solution (HBSS, Sigma). Cortices were dissected out and digested for 15 minutes at 37° C. in 0.25% trypsin (Gibco) and 0.1% DNase (Sigma) in HBSS. After digestion, the tissue was washed three times in ice-cold HBSS and then triturated with fire-polished Pasteur pipettes of decreasing pore size in HBSS with 0.1% DNase. After centrifugation for 10 minutes at 1,000 rpm, the cell pellet was resuspended in plating media consisting of Neurobasal medium (Gibco) supplemented with B27 (Gibco), L-glutamine (500 μM), and L-glutamate (25 μM). Cells were then counted and plated at 0.75×10⁵ cells/well in twelve well plates, each well containing one 10 mm glass coverslip (Assistent) that had been preincubated overnight in 40 μg/ ml poly-D-lysine (Fischer) and 2.5 μg/ml laminin (Fischer), rinsed three times in water, and then incubated in plating medium. After four days in vitro (DIV), half the plating media was replaced with feeding medium (plating medium minus L-glutamate). Cultures were maintained in a humidified 37° C. incubator with a 5% CO₂ atmosphere.

After 6 DIV, cortical neurons were washed three times with Neurobasal medium without supplements, then incubated for 12 hours in the unsupplemented medium with or without 5 μg/ml purified CPG15 protein. After an additional 12 hours in feeding medium, cells were fixed in 4% formaldehyde/PBS for 30 minutes at 4° C. before Hoechst staining or immunocytochemistry. Fixed cells were incubated 30 minutes with Hoechst 33342 (1:1000, in PBS, Sigma), rinsed three times in PBS and mounted onto slides with Fluoromount G (Southern Biotechnology). For immunocytochemistry, fixed cells were washed with PBS for five minutes, then permeabilized with 0.3% Triton X-100 for five minutes at 4° C. Neurons were washed again with PBS, incubated with blocking solution (10% goat serum, 0.1% Triton X-100 in PBS) for one hour at 4° C., and then incubated with an anti-cleaved Caspase3 antibody (1:100, Cell Signaling Technology) in blocking solution overnight at 4° C. After rinsing three times with PBS, an anti-rabbit secondary antibody coupled to rhodamine (1:500, Jackson Immuno Research) was added for one hour at room temperature. Chromatin staining with Hoechst was done simultaneously, and neurons were rinsed and mounted as described above.

For quantification, fragmented apoptotic nuclei as well as healthy nuclei were counted blind to experimental treatment using a fluorescence microscope with UV filter setting for the Hoechst staining (excitation 330-380; emission 420) and rhodamine settings for visualizing the antibody against cleaved caspase3 (excitation 528-553; emission 600-660). Treatments were repeated in three independent experiments with two coverslips per treatment in each experiment. Each data point represents the mean of 500-600 cells, counted in 40-50 different fields per coverslip. The percent apoptotic cells was calculated based on the number of condensed/fragmented nuclei divided by the total number of nuclei. Comparisons between groups were analyzed using a student's t-test.

For some of the experiments described below, the minimal media also contained s-CPG15 containing supernatant from 293T cells transfected with CPG15-FLAG IRES EGFP. The cells were then returned to full neuronal media and incubated for twelve hours. Chromatin DNA was then stained with Hoechst 33342 and apoptotic neurons were counted.

### Example 6

### Supernatants Containing s-CPG15 Prevents Starvation-Induced Apoptosis

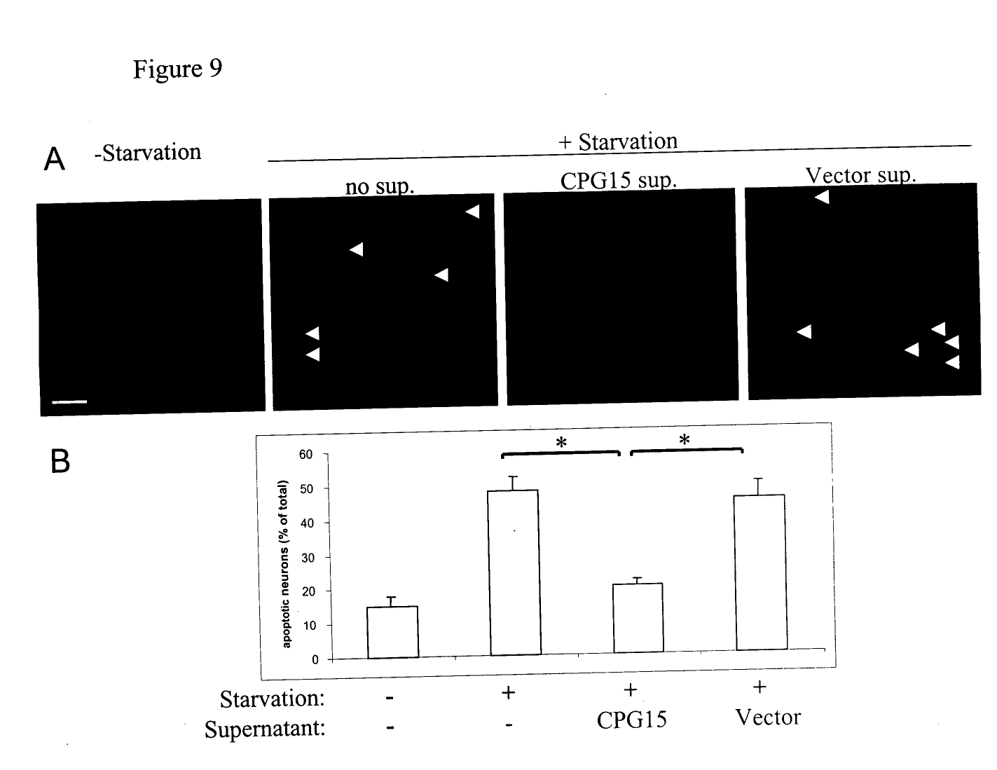

In order to determine if soluble CPG15 found in the supernatant could act as a survival factor to protect against apoptosis, primary hippocampal neurons were cultured for six days in normal feeding media and then starved as described in Example 5 in the absence or presence of supernatant from 293T cells overexpressing CPG15-FLAG or from 293T cells transfected with vector alone. After twelve hours, medium was changed back to normal feeding media for an additional twelve hours. Neurons were then stained with Hoechst 33342 and apoptotic nuclei were counted (see FIGS. 9A and 9B). These results demonstrate that the supernatant taken from cells overexpressing CPG15-FLAG could protect against apoptosis and strongly suggested that the soluble CPG15 present in this supernatant was the factor responsible for this activity.

### Example 7

### Purified s-CPG15 Prevents Starvation-Induced Apoptosis

The presence of soluble CPG15 in brain extracts at early developmental times, and the ability of the supernatant taken from cells overexpressing CPG15 to protect against apoptosis, led us to test whether it may serve as a survival factor that protects against apoptosis. We assessed whether soluble CPG15 affinity purified from supernatants of CPG15-FLAG transfected HEK293T cells was capable of preventing cortical neurons from undergoing programmed cell death associated with growth factor deprivation.

HEK293T cells were grown to 80 -90% confluence in 100 mm culture dishes containing 15 ml of media (10% calf serum, 50 U Penicillin, 50 μg Streptomycin, 4 mM L-glutamate in Dulbeccos Modified Eagles medium (BioWhittaker)), then transfected with 8 μg of the pIRES EGFP-CPG15 FLAG plasmid (FIG. 4A) using Lipofectamine2000 (Invitrogen). The medium was harvested four days later and debris removed by centrifugation (3,000 rpm, 15 minutes, 4° C.). The medium was then incubated with 40 μl anti-FLAG antibody coupled to agarose (EZview Red ANTI-FLAG M2 Affinity Gel, Sigma) for 12 hrs at 4° C. on a rotator. The agarose was pelleted by centrifugation for 10 minutes at 2,000 rpm and washed three times with TBS. The tagged CPG15 protein was eluted from the anti-FLAG antibody by incubation with 30 μg of 3× FLAG peptide (Sigma) diluted in 200 pl TBS for four hours at 4° C., then recovered by centrifugation for five minutes at 2,000 rpm. Protein concentration in the supernatant was determined by the Bradford assay.

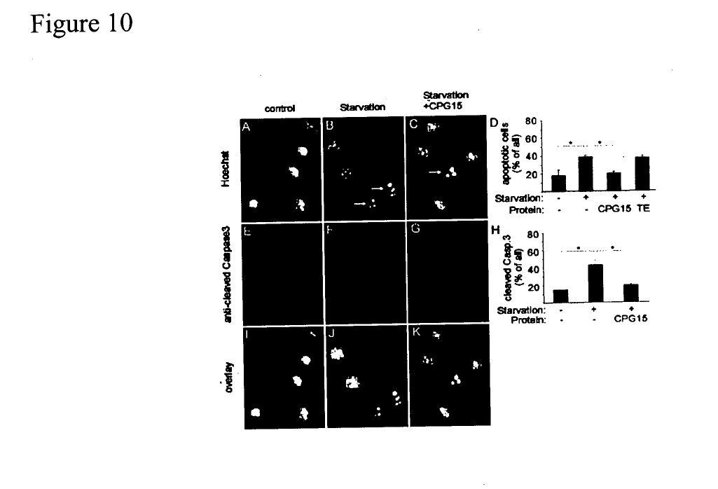

Using Hoechst 33324 staining to identify cells with fragmented nuclei, we counted the number of apoptotic neurons in untreated cortical cultures, and in cultures after growth factor deprivation (starvation), with or without addition of purified CPG15 (FIGS. 10A-10D). Growth factor deprivation more than doubled the level of apoptosis in the cultures, increasing the percentage of apoptotic neurons from approximately 15% to 40%. This increased apoptosis could be completely prevented by the addition of soluble CPG15, but not by addition of the affinity column elution buffer (FIG. 10D). To confirm that CPG15 was indeed rescuing neurons from apoptotic rather than necrotic cell death, the treated and control cultures were immunostained with an antibody specific to the large subunit of activated caspase 3. Caspase 3 is a key component of the apoptotic pathway in brain development, and its activation requires proteolytic cleavage of the inactive zymogen into activated p17 and p12 subunits. All neurons seen by Hoechst staining to contain pyknotic nuclei also stained positive for the p17 subunit of caspase 3 (FIGS. 10E-10K). Independent quantification of immunostained neurons positive for cleaved caspase 3, showed that starvation more than doubled the number of neurons in culture containing activated caspase3. CPG15 application completely prevented the increased caspase 3 activation induced by starvation (FIG. 10H). We conclude that soluble CPG15 protects cortical neurons from apoptosis by preventing activation of caspase pathways induced by growth factor deprivation.

### Example 8

### Depletion of Endogenous CPG15 In Vivo

Since CPG15 promotes survival of cortical neurons in vitro, we predicted that depletion of endogenous CPG15 at early developmental times would increase apoptosis of cortical neurons in vivo. To examine this hypothesis we utilized the RNAi (RNA interference) technique to knockdown CPG15 expression, combined with the lentivirus delivery system (see Lois et al., supra and Rubinson et al., supra). We generated a lentivirus expressing a cpg15-shRNA (small hairpin RNA) driven by the RNA-polymerase III promoter. shRNAs are processed by the protein Dicer to small interfering RNAs (siRNAs) that guide the cleavage and elimination of their specific cognate mRNAs.

Five different cDNA sequences spanning the cpg15 core domain were synthesized fused to a loop region, then annealed to their antisense sequences and cloned separately into pSilencer 1.0-U6 plasmid (Ambion) downstream of the U6 promoter. To test the effectiveness of the cpg15-shRNAs in reducing CPG15 levels, HEK293T cells were separately co-transfected with each one of the pSilencer-cpg15-shRNA plasmids together with the pIRES-EGFP-CPG15-FLAG plasmid at a 40:1 ratio using Lipofectamine 2000 (Invitrogen). CPG15-IRES-EGFP mRNA knockdown was determined by reduced expression of EGFP. The two most effective small hairpin sequence were: (GGGCTTTTCAGACTGTTTG, SEQ ID NO: 2) and GTTGAACGGCAGATATATT, SEQ ID NO: 3). The shRNA of SEQ ID NO: 2 was chosen and then amplified with its upstream U6 promoter by PCR and sub dloned into the pFUGW lentivirus transfer vector. The CPG15-FLAG-IRES-EGFP cDNA was subcloned into pFUGW downstream of the ubiquitin promoter. Lentivirus production, concentration, and titer determination were performed as described (Lois et al., supra). Typical titers for in vitro experiments were 1×10⁶, and for in vivo injections 5×10⁶ to 20×10⁶.

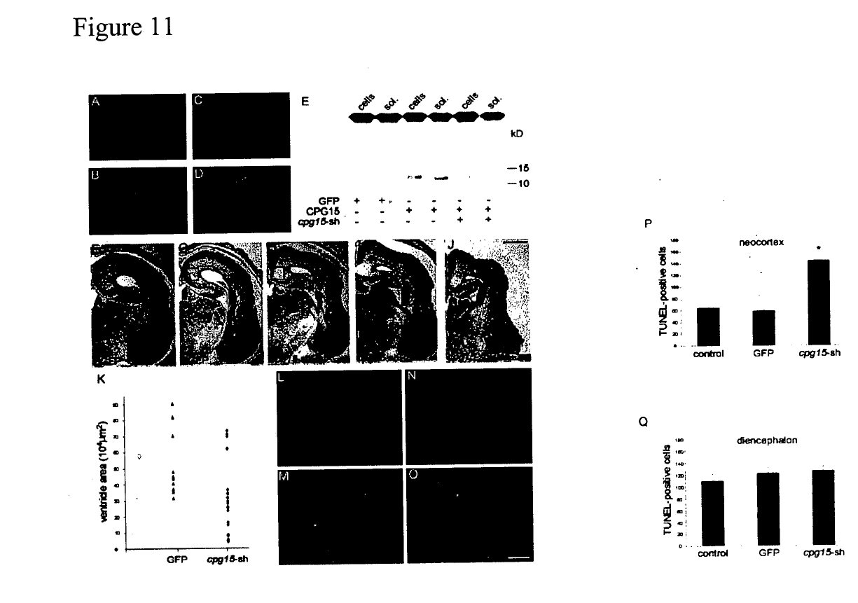

When primary cortical cultures were co-infected with cpg15-shRNA-lentivirus and a CPG15-FLAG-lentivirus, levels of CPG15-FLAG protein were severely reduced as seen both by immunohistochemistry and western blotting (FIGS. 11A-11E), demonstrating that the cpg15-shRNA-lentivirus is effective in reducing cellular levels of CPG15.

For the in vivo experiments, the cpg15-shRNA-lentivirus or a control EGFP-lentivirus were delivered into the developing brain by direct injection into the ventricles of E15 embryos. For these experiments, neurons and progenitors were infected by injecting 2-2.5 μl of lentivirus into the lateral ventricle of E15 brains. Surgery and injection procedures were as described elsewhere (Walsh et al., Science 255:317-320, 1992). Timed-pregnant Sprague-Dawley rats were purchased from Taconic (Germantown, N.Y.). Pregnancies were timed with day of plug detection as E1. Birth usually occurred on E23.

Embryos were harvested at E22 and their brains sectioned. Alternate sections were Nissl stained for histology or TUNEL stained for detection of apoptotic neurons as follows.

Animals were euthanized at E22, seven days after viral injection. Brains were removed and submerged in 4% paraformaldehyde in PBS overnight at 4° C., transferred to 30% sucrose in PBS at 4° C. until they sank, then frozen and sectioned at 20μm using a cryostat. Every sixth section was stained for Nissl and used to match treated brains with controls. Sections at equivalent levels on the anterior/posterior axis were processed for TUNEL.

TUNEL staining on frozen brain sections was performed as described by the manufacturer using the Roche In Situ Cell Death Detection Kit, TMR red (Penzberg, Germany). Immunohistochemistry was performed using a rabbit polyclonal antibody against EGFP (1:1000, Abcam). The total number of TUNEL positive cells present in the neocortex and the diencephalon of three control, three GFP and four cpg15-shRNA infected brains were quantified. Six to twelve sections were analyzed from each brain. Statistical significance was determined by student's t-test.

Examination of the Nissl stained sections revealed that when compared with uninfected or EGFP-lentivirus infected brains (FIGS. 11F, 11G) the brains infected with the cpg15-shRNA-lentivirus displayed moderate to severe brain abnormalities (FIGS. 11H-11J), depending on the levels of infection. Overall cpg15-shRNA brains showed reduced ventricle size (FIG. 11K) and somewhat contracted cortices. Numbers of apoptotic neurons were assayed by TUNEL staining in the neocortex, where CPG15 is highly expressed during embryonic development and in the diencephalon where CPG15 expression is low in early development (FIG. 3). Interestingly, when compared to GFP infected brains the cpg15-shRNA brains, showed a dramatic increase in the number of apoptotic cells in the neocortex but not in the diencephalon. Similar to the gross morphological abnormalities, levels of apoptosis in the cpg15-shRNA infected brains correlated with levels of infection. These results demonstrate that decreasing the level of endogenous CPG15 during embryonic development resulted in increased apoptosis and diminished survival of cortical neurons. Reduced neuronal number likely causes shrinkage of the cortical plate and its contraction around the lateral ventricles as seen in the cpg15-shRNA-lentivirus infected brains. Thus, we demonstrated that shRNA delivered by a lentivirus can be effectively used as a tool for investigating the function of specific proteins in the developing brain in vivo and we confirmed an important role of CPG15 in the survival of neurons during early development in the neocortex.

These examples demonstrate that soluble CPG15 appears to be the predominant functional form of the protein and that it functions as both a survival factor that can rescue from cell death, and as a growth and differentiation factor that affects process outgrowth and synaptic maturation.

### Other Embodiments

All publications and patents mentioned in this specification are herein incorporated by reference to the same extent as if each individual publication or patent was specifically and individually incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed is:

1. A method of treating or preventing a condition of excessive cell death in a subject, said method comprising administering to said subject a soluble CPG15 (s-CPG15) compound having s-CPG15 biological activity in an amount and for a time sufficient to prevent, reduce, or eliminate the symptoms of said condition.

2. The method of claim 1, wherein said s-CPG15 lacks either a signal sequence or a GPI linkage sequence.

3. The method of claim 2, wherein said s-CPG15 lacks a signal sequence and a GPI linkage sequence.

4. The method of claim 3, wherein said soluble CPG15 lacking a signal sequence and a GPI linkage sequence has the in vitro biological activity of a CPG15 protein wherein
a) the signal sequence and the GPI linkage sequence of said CPG15 protein have been cleaved;
b) said CPG15 protein has been bound to a cell membrane; and
c) said CPG15 protein has been released from the cell.

5. The method of claim 3, wherein said s-CPG15 comprises the sequence of SEQ ID NO: 1.

6. The method of claim 3, wherein said s-CPG15 comprises a post-translational modification.

7. The method of claim 6, wherein said post-translational modification comprises the attachment of a membrane component to said s-CPG15.

8. A method of reducing or preventing cell death, said method comprising administering to a cell s-CPG15 in an amount and for a time sufficient to reduce or prevent said cell death.

9. The method of claim 8, wherein said s-CPG15 lacks either a signal sequence or a GPI linkage sequence.

10. The method of claim 9, wherein said s-CPG15 lacks a signal sequence and a GPI linkage sequence.

11. The method of claim 10, wherein said soluble CPG15 lacking a signal sequence and a GPI linkage sequence has the in vitro biological activity of a CPG15 protein wherein
a) the signal sequence and the GPI linkage sequence of said CPG15 protein have been cleaved;
b) said CPG15 protein has been bound to a cell membrane; and
c) said CPG15 protein has been released from the cell.

12. A method of promoting the survival or differentiation of a cell comprising administering to said cell s-CPG15 for a time and in an amount to promote the survival or differentiation of said cell.

13. The method of claim 12, wherein said soluble CPG15 lacking a signal sequence and a GPI linkage sequence has the in vitro biological activity of a CPG15 protein wherein
a) the signal sequence and the GPI linkage sequence of said CPG15 protein have been cleaved;
b) said CPG15 protein has been bound to a cell membrane; and
c) said CPG15 protein has been released from the cell.

14. The method of claim 12, wherein said survival or differentiation of said cell is increased by at least 5% as compared to a control cell not treated by said method.

15. A composition of matter comprising a purified polypeptide having s-CPG15 biological activity.

16. The composition of claim 15, wherein said s-CPG15 lacks either a signal sequence or a GPI linkage sequence.

17. The composition of claim 16, wherein said s-CPG15 lacks a signal sequence and a GPI linkage sequence.

18. The composition of claim 17, wherein said soluble CPG15 lacking a signal sequence and a GPI linkage sequence has the in vitro biological activity of a CPG15 protein wherein
a) the signal sequence and the GPI linkage sequence of said CPG15 protein have been cleaved;
b) said CPG15 protein has been bound to a cell membrane; and
c) said CPG15 protein has been released from the cell.

19. The composition of claim 17, wherein said s-CPG15 comprises the sequence of SEQ ID NO: 1.

20. The composition of claim 17, wherein said s-CPG15 comprises a posttranslational modification.

21. A method of treating or preventing a condition of undesirable cell survival in a subject, said method comprising administering to said subject a purified antibody or antigen-binding fragment that specifically binds a polypeptide having s-CPG15 biological activity, wherein said administering is in an amount and for a time sufficient to prevent, reduce, or eliminate the symptoms of said condition.

22. The method of claim 21, wherein said condition is selected from the group consisting of cancer, tumor-associated angiogenesis, or immune system conditions.

23. A method of treating or preventing a condition of undesirable cell survival in a subject, said method comprising administering to said subject a double-stranded RNA that is complementary to an mRNA sequence encoding a protein having s-CPG15 biological activity, wherein said administering is sufficient to reduce or inhibit the expression of s-CPG15.

24. The method of claim 23, wherein said condition is selected from the group consisting of cancer, tumor-associated angiogenesis, or immune system conditions.

25. The method of claim 23, wherein said double-stranded RNA is processed into small interfering RNAs (siRNAs) 19 to 25 nucleotides in length.

26. The method of claim 23, wherein said double stranded RNA is made from a short hairpin RNA (shRNA).

27. The method of claim 26, wherein said shRNA comprises the sequence of SEQ ID NO: 2.

28. The method of claim 26, wherein said shRNA comprises the sequence of SEQ ID NO: 3.

29. A method of treating or preventing a condition of undesirable cell survival in a subject, said method comprising administering to said subject a nucleic acid encoding a truncated form of CPG15, wherein said administering is sufficient to reduce or inhibit the biological activity of s-CPG15.

30. The method of claim 29, wherein said condition is selected from the group consisting of cancer, tumor-associated angiogenesis, or immune system conditions.

31. A method of treating or preventing a condition of undesirable cell survival in a subject, said method comprising administering to said subject a compound comprising a truncated form of CPG15 protein, wherein said administering is sufficient to reduce or inhibit the biological activity of s-CPG15.

32. The method of any one of claims 21, 23, 29, and 31, wherein said undesirable cell survival is reduced by at least 5%.

33. A method of enhancing cell death, said method comprising administering to a cell an antibody or antigen-binding fragment that specifically binds s-CPG15.

34. A method of enhancing cell death, said method comprising administering to a cell a double-stranded RNA that is complementary to an mRNA sequence encoding a protein having s-CPG15 biological activity.

35. The method of claim 34, wherein said double-stranded RNA is processed into small interfering RNAs (siRNAs) 19 to 25 nucleotides in length.

36. The method of claim 35, wherein said siRNA comprises the sequence of SEQ ID NO: 2.

37. The method of claim 35, wherein said siRNA comprises the sequence of SEQ ID NO: 3.

38. A method of enhancing cell death, said method comprising administering to a cell a nucleic acid encoding a truncated form of CPG15, wherein said administering is sufficient to reduce or inhibit the biological activity of s-CPG15.

39. A method of enhancing cell death, said method comprising administering to a cell a compound comprising a truncated form of CPG15 protein, wherein said administering is sufficient to reduce or inhibit the biological activity of s-CPG15.

40. The method of any one of claims 33, 34, 39, and 38, wherein said cell death is increased by at least 5%.

41. A composition of matter comprising a purified antibody or antigen-binding fragment thereof that specifically binds s-CPG15.

42. A composition of matter comprising an siRNA that is complementary to an MRNA sequence encoding s-CPG15.

43. A method of manufacturing s-CPG15 comprising expressing said s-CPG15 protein in a population of cells and isolating from the supernatant of said cell population said s-CPG15.

44. The method of claim 43, wherein said s-CPG15 is at least 85% pure.

45. The method of claim 43, wherein said s-CPG15 comprises less than 20% CPG15.

46. The method of claim 43, wherein said s-CPG15 comprises a posttranslational modification.

47. The method of claim 46, wherein said post-translational modification comprises the attachment of a membrane component to said s-CPG15.

48. The method of claim 43, wherein said cells are neuronal cells or hippocampal cells.

49. The method of claim 43, wherein said cells are selected from the group consisting of COS cells, CV-1 cells, L cells, C127 cells, 3T3 cells, CHO cells, HeLA cells, 293 cells, 293T cells, and BHK cells.

50. A method of identifying a candidate compound that modulates cell death, cell survival, or cellular differentiation pathways, said method comprising the steps of
(a.) mixing s-CPG15 with a test mixture,
(b.) identifying a candidate compound in said mixture that interacts with said s-CPG15.

51. The method of claim 50, further comprising the step after step (a) of incubating said s-CPG15/test mixture with an insoluble affinity support reagent that specifically binds s-CPG15.

52. The method of claim 51, further comprising the step of recovering said affinity support reagent bound to CPG15.

53. The method of claim 50, wherein said mixture is a cell lysate.

54. The method of claim 50, wherein said mixture is a lysate from a tissue.

55. The method of claim 50, wherein said modulation comprises an increase said cell death, cell survival, or cellular differentiation pathways.

56. The method of claim 55, wherein said increase is at least 5%.

57. The method of claim 50, wherein said modulation comprises a decrease in said cell death, cell survival, or cellular differentiation pathways.

58. The method of claim 57, wherein said decrease is at least 5%.


SEQUENCE LISTING (3 sequences)

SEQ ID NO: 1 - protein, 87 aa, Homo sapiens
AGKCDAVFKGFSDCLLKLGDSMANYPQGLDDKTNIKTVCTYWEDFHSCTVTALTDCQEGAKDMWDKLRKESKNLNIQGSLFELCGSG

SEQ ID NO: 2 - RNA, 19 nt, Homo sapiens
gggcuuuucagacuguuug

SEQ ID NO: 3 - RNA, 19 nt, Homo sapiens
guugaacggcagauauauu